SEQUENCE LISTING (21 sequences)

SEQ ID NO: 1 - protein, 72 aa, Artificial Sequence
  Linker
GGSSGSGSGSTGTSSSGTGTSAGTTGTSASTSGSGSGGGGGSGGGGSAGGTATAGASSGSGSSGSSSSGGTG

SEQ ID NO: 2 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 3 - protein, 227 aa, Homo sapiens; identical to SEQ ID NO: 2

SEQ ID NO: 4 - protein, 219 aa, Artificial Sequence
  Ibalizumab-A12P light chain
DIVMTQSPDSLPVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 227 aa, Artificial Sequence
  Ibalizumab-A12P heavy chain
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGSR

SEQ ID NO: 6 - protein, 213 aa, Artificial Sequence
  10E8.v4 light ch
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 7 - protein, 235 aa, Artificial Sequence
  10E8.v4 heavy chain
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCS

SEQ ID NO: 8 - protein, 204 aa, Artificial Sequence
  N49P7 light chain
QSALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 9 - protein, 230 aa, Artificial Sequence
  N49P7 heavy chain
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSR

SEQ ID NO: 10 - protein, 219 aa, Artificial Sequence
  PGDM1400 light chain
DFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 247 aa, Artificial Sequence
  PGDM1400 heavy chain
QAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSR

SEQ ID NO: 12 - protein, 90 aa, Homo sapiens
MSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 13 - protein, 85 aa, Homo sapiens
GKTPDAMKAAMALEKKLNQALLDLHALGSARTDPHLCDFLETHFLDEEVKLIKKMGDHLTNLHRLGGPEAGLGEYLFERLTLRHD

SEQ ID NO: 14 - protein, 25 aa, Artificial Sequence
  Linker
ASTASSASSGGGGGGSGGSGGSGGS

SEQ ID NO: 15 - protein, 20 aa, Artificial Sequence
  Linker
GGSGGSGGSGGSGGGASGGS

SEQ ID NO: 16 - protein, 758 aa, Artificial Sequence
  PGDM1400-hFerr
DFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSGSSGSSSSGGTGQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSRASTASSASSGGGGGGSGGSGGSGGSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHDGGSGGSGGSGGSGGGASGGS

SEQ ID NO: 17 - protein, 643 aa, Artificial Sequence
  Fc-N-hFerr LS
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGKGGSSGSGSGSTGTSSSGTGTSAGTTGTSASTSGSGSGGGGGSGGGGSAGGTATAGASSGSGSSGSSSSGGTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGKSRASTASSASSGGGGGGSGGSGGSGGSMSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 18 - protein, 639 aa, Artificial Sequence
  N49P7-C-hFerr
QSALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGSSGSGSGSTGTSSSGTGTSAGTTGTSASTSGSGSGGGGGSGGGGSAGGTATAGASSGSGSSGSSSSGGTGADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSRASTASSASSGGGGGGSGGSGGSGGSGKTPDAMKAAMALEKKLNQALLDLHALGSARTDPHLCDFLETHFLDEEVKLIKKMGDHLTNLHRLGGPEAGLGEYLFERLTLRHDGGSGGSGGSGGSGGGASGGS

SEQ ID NO: 19 - protein, 648 aa, Artificial Sequence
  Ibalizumab-A12P-C-hFerr
DIVMTQSPDSLPVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSSGSGSGSTGTSSSGTGTSAGTTGTSASTSGSGSGGGGGSGGGGSAGGTATAGASSGSGSSGSSSSGGTGQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGSRASTASSASSGGGGGGSGGSGGSGGSGKTPDAMKAAMALEKKLNQALLDLHALGSARTDPHLCDFLETHFLDEEVKLIKKMGDHLTNLHRLGGPEAGLGEYLFERLTLRHDGGSGGSGGSGGSGGGASGGS

SEQ ID NO: 20 - protein, 651 aa, Artificial Sequence
  10E8.v4-C-hFerr
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGSSGSGSGSTGTSSSGTGTSAGTTGTSASTSGSGSGGGGGSGGGGSAGGTATAGASSGSGSSGSSSSGGTGEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSRASTASSASSGGGGGGSGGSGGSGGSGKTPDAMKAAMALEKKLNQALLDLHALGSARTDPHLCDFLETHFLDEEVKLIKKMGDHLTNLHRLGGPEAGLGEYLFERLTLRHDGGSGGSGGSGGSGGGASGGS

SEQ ID NO: 21 - protein, 710 aa, Artificial Sequence
  scFab 10E8
YELTQETGVSVALGRTVTITCRGDSLRSHYASWYQKKPGQAPILLFYGKNNRPSGVPDRFSGSASGNRASLTISGAQAEDDAEYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGSSGSGSGSTGENLYFQGSAGTTGTSASTSGYPYDVPDYAGGGGSAGGTATLEVLFQGPSSGSSSSGGTGEVQLVESGGGLVKPGGSLRLSCSASGFDFDNAWMTWVRQPPGKGLEWVGRITGPGEGWSVDYAAPVEGRFTISRLNSINFLYLEMNNLRMEDSGLYFCARTGKYYDFWSGYPPGEEYFQDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSRGGGGGSGGSGGSGGSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

(19) United States
(12) Patent Application Publication
(10) Pub. No.: US 2023/0145060 A1
(43) Pub. Date: May 11, 2023

(54) MULTI-VALENT AND MULTI-SPECIFIC NANOPARTICLE PLATFORMS AND METHODS

(71) Applicants: The Hospital for Sick Children, Toronto, Ontario (CA); The Governing Council of the University of Toronto, Toronto, Ontario (CA)

(72) Inventors: Jean-Philippe Julien, Toronto, Ontario (CA); Edurne Rujas Diez, Toronto, Ontario (CA); Bebhinn Treanor, Toronto, Ontario (CA); Tiantian Zhao, Toronto, Ontario (CA)

(21) Appl. No.: 17/631,588

(22) PCT Filed: Jul. 31, 2020
(86) PCT No.: PCT/CA2020/051061

§ 371 Date: Jan. 31, 2022

Related U.S. Application Data
(60) Provisional application No. 62/881,899, filed on Aug. 1, 2019.

(51) Int. Cl. A61K 39/21; A61K 47/64; A61K 47/68; A61K 39/385; A61K 49/00; A61P 31/18; C12P 21/02

(52) U.S. Cl. CPC A61K 39/21; A61K 47/64; A61K 47/6803; A61K 39/385; A61K 49/0002; A61P 31/18; C12P 21/02; A61K 2039/6056

(57) ABSTRACT

A fusion protein comprises a first nanocage monomer subunit of a nanocage monomer; and a bioactive moiety linked to the first nanocage monomer subunit; wherein the fusion protein self-assembles with a protein comprising a second nanocage monomer subunit to form a nanocage monomer.

### STATEMENT REGARDING ELECTRONIC FILING OF A SEQUENCE LISTING

A Sequence Listing in ASCII text format, submitted under 37 C.F.R. 1.821, entitled 1360-33_ST25, 58,643 bytes in size, generated on Jan. 28, 2022, and filed via EFS-Web, is provided in lieu of a paper copy. This Sequence Listing is hereby incorporated herein by reference into the specification for its disclosures.

### FIELD

The present invention relates to nanoparticles. In particular, the present invention relates to nanoparticle subunit fusion proteins, vaccines, prophylactics and therapeutics comprising the nanoparticles, and related compositions and methods.

### BACKGROUND

Nanoparticles have contributed to advancements in various disciplines. Their use has the potential to confer targeted delivery and allows the engineering of ordered micro-arrays, slow release and caged micro-environments for catalytic processes.

For the fabrication of nanoparticles that contain sensitive and metastable proteins, protein self-assembly is an attractive method. Indeed, self-assembled nanoparticles form under physiological conditions through non-covalent interactions and reliably yield uniform and often symmetric nanocapsules or nanocages. Self-assembling protein nanoparticles possess three distinct surfaces that can all be tweaked to convey added functionalities: exterior, interior and inter-subunits surfaces.

Fusion proteins comprising self-assembling proteins have been described. For example, it is known to display antigens on the exterior surface of assembled nanocages for use as vaccines.

A need exists for improved compositions and methods involving nanocages.

### SUMMARY OF THE INVENTION

Described herein, in aspects, are fusion proteins and self-assembling nanocages, as well as related compositions and methods, that allow presentation and tuning of multiple cargos on a single nanoparticle, for example, multiple copies of the same cargo and/or different cargos. In some embodiments, the presently disclosed fusion proteins, nanocages, compositions, and methods allow for control of ratios of different cargo molecules, for example, to optimize the self-assembled nanocage for a particular therapeutic and/or prophylactic purpose.

In accordance with an aspect, there is provided a fusion protein comprising:

a first nanocage monomer subunit of a nanocage monomer; and

a bioactive moiety linked to the first nanocage monomer subunit;

wherein the fusion protein self-assembles with a protein comprising a second nanocage monomer subunit to form a nanocage monomer.

In an aspect, the bioactive moiety decorates the interior and/or exterior surface of the assembled nanocage.

In an aspect, the bioactive moiety comprises an antibody or fragment thereof, an antigen, a detectable moiety, a pharmaceutical agent, a diagnostic agent, or combinations thereof.

In an aspect, the antibody or fragment thereof comprises an Fc fragment.

In an aspect, the Fc fragment is an IgG1 Fc fragment.

In an aspect, the Fc fragment comprises one or more mutations, such as LS, YTE, LALA, and/or LALAP, that modulate the half-life of the fusion protein from, for example, minutes or hours to several days, weeks, or months.

In an aspect, the antibody or fragment thereof comprises a Fab fragment.

In an aspect, the antibody or fragment thereof comprises a scFab fragment, a scFv fragment, or a sdAb fragment.

In an aspect, the antibody or fragment thereof comprises a heavy and/or light chain of a Fab fragment.

In an aspect, the antibody or fragment thereof comprises both a light chain and a heavy chain, or in the case of a Fc fragment, a first and a second chain, optionally separated by a linker.

In an aspect, the linker comprises or consists of a sequence at least 70% (such as at least 75%, 80%, 85%, 90%, 95%, 96%, 97%, 98%, 99%, or 100%) identical to:

GGSSGSGSGSTGTSSSGTGTSAGTTGTSASTSGSGSGGGGGSGGGGSAG
GTATAGASSGSGSSGSSSSGGTG.

In an aspect, the fusion protein is in association with a separately produced Fab light chain and/or heavy chain.

In an aspect, the antibody or fragment thereof binds specifically to an antigen associated with an antibody-preventable and/or antibody-treatable condition.

In an aspect, the antigen is associated with an infectious agent, including a virus (e.g., HIV, including HIV-1, influenza, RSV, rotavirus), bacteria (e.g., TB, C. difficile) parasite (e.g., malaria), fungus, or yeast, a cancer (e.g., CD19, CD22, CD79, BCMA, or CD20), including solid and liquid cancers, or an immune disease, including an autoimmune disease.

In an aspect, the antigen is associated with HIV-1 and the antibody or fragment thereof comprises, for example, lbalizumab-A12P, 10E8, 10E8.v4, N49P7, PGDM1400, 10-1074, VRC01, or combinations thereof.

In an aspect, the antibody or fragment thereof comprises or consists of a sequence at least 70% (such as at least 75%, 80%, 85%, 90%, 95%, 96%, 97%, 98%, 99%, or 100%) identical to:

Fc chain 1:
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRW
QQGNVFSCSVLHEALHSHYTQKSLSLSPGK;
Fc chain 2:
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRW
QQGNVFSCSVLHEALHSHYTQKSLSLSPGK;
Ibalizumab-A12P light chain:
DIVMTQSPDSLPVSLGERVTMNCKSSQSLLYSTNQKNYLAWYQQKPGQS
PKLLIYWASTRESGVPDRFSGSGSGTDFTLTISSVQAEDVAVYYCQQYY
SYRTFGGGTKLEIKRTVAAPSVFIFPPSDEQLKSGTASWCLLNNFYPRE
AKVQWKVDNALQSGNSQESVTEQDSKDSTYSLSSTLTLSKADYEKHKVY
ACEVTHQGLSSPVTKSFNRGEC;
Ibalizumab-A12P heavy chain:
QVQLQQSGPEWKPGASVKMSCKASGYTFTSYVIHWVRQKPGQGLDWIGY
INPYNDGTDYDEKFKGKATLTSDTSTSTAYMELSSLRSEDTAVYYCARE
KDNYATGAWFAYWGQGTLVTVSSASTKGPSVFPLAPCSRSTSESTAALG
CLVKDYFPEPVTVSWNSGALTSGVHTFPAVLQSSGLYSLSSVVTVPSSS
LGTKTYTCNVDHKPSNTKVDKRVESKYGSR;
10E8.v4 light chain:
SELTQDPAVSVALKQTVTITCRGDSLRSHYASWYQKKPGQAPVLLFYGK
NNRPSGIPDRFSGSASGNRASLTITGAQAEDEADYYCSSRDKSGSRLSV
FGGGTKLTVLSQPKAAPSVTLFPPSSEELQANKATLVCLISDFYPGAVT
VAWKADSSPVKAGVETTTPSKQSNNKYAASSYLSLTPEQWKSHRSYSCQ
VTHEGSTVEKTVAPTEC;
10E8.v4 heavy chain:
EVRLVESGGGLVKPGGSLRLSCSASGFDFDNAWMTWVRQPPGKGLEWVG
RITGPGEGWSVDYAESVKGRFTISRDNTKNTLYLEMNNVRTEDTGYYFC
ARTGKYYDFWSGYPPGEEYFQDWGQGTLVIVSSASTKGPSVFPLAPSSK
STSGGTAALGCLVKDYFPEPVTVSWNSGALTSGVHTFPAVLQSSGLYSL
SSVVTVPSSSLGTQTYICNVNHKPSNTKVDKKVEPKSCSR;
N49P7 light chain:
QSALTQPRSVSASPGQSVTISCTGTHNLVSWCQHQPGRAPKLLIYDFNK
RPSGVPDRFSGSGSGGTASLTITGLQDDDDAEYFCWAYEAFGGGTKLTV
LGQPKAAPSVTLFPPSSEELQANKATLVCLISDFYPGAVTVAWKADSSP
VKAGVETTTPSKQSNNKYAASSYLSLTPEQWKSHRSYSCQVTHEGSTVE
KTVAPTEC;
N49P7 heavy chain:
ADLVQSGAWKKPGDSVRISCEAQGYRFPDYIIHWIRRAPGQGPEWMGWM
NPMGGQVNIPWKFQGRVSMTRDTSIETAFLDLRGLKSDDTAVYYDRSNG
SGKRFESSNWFLDLWGRGTAVTIQSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDSR;
PGDM1400 light chain:
DFVLTQSPHSLSVTPGESASISCKSSHSLIHGDRNNYLAWYVQKPGRSP
QLLIYLASSRASGVPDRFSGSGSDKDFTLKISRVETEDVGTYYCMQGRE
SPWTFGQGTKVDIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC;
PGDM1400 heavy chain:
QAQLVQSGPEVRKPGTSVKVSCKAPGNTLKTYDLHWVRSVPGQGLQWMG
WISHEGDKKVIVERFKAKVTIDWDRSTNTAYLQLSGLTSGDTAVYYCAK
GSKHRLRDYALYDDDGALNWAVDVDYLSNLEFWGQGTAVTVSSASTKGP
SVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCD
SR;

combinations thereof.

In an aspect, the antibody or fragment thereof is conjugated to or associated with a further moiety, such as an antigen, a detectable moiety (e.g., a small molecule, fluorescent molecule, radioisotope, or magnetic particle), a pharmaceutical agent, a diagnostic agent, or combinations thereof.

In an aspect, the antibody or fragment thereof comprises an antibody-drug conjugate.

In an aspect, the antigen is associated with a vaccine-preventable and/or vaccine-treatable condition.

In an aspect, the antigen is associated with an infectious agent, including a virus, bacteria, parasite, fungus, or yeast, a cancer, including solid and liquid cancers, or an immune disease, including an autoimmune disease.

In an aspect, the detectable moiety comprises a fluorescent protein, such as GFP, EGFP, Ametrine, and/or a flavin-based fluorescent protein, such as a LOV-protein, such as iLOV.

In an aspect, the pharmaceutical agent comprises a small molecule, peptide, lipid, carbohydrate, or toxin.

In an aspect, from about 3 to about 100 nanocage monomers, such as 24, 32, or 60 monomers, or from about 4 to about 200 nanocage monomer subunits, such as 4, 6, 8, 10, 12, 14, 18, 20, 22, 24, 26, 28, 30, 32, 34, 36, 38, 40, 42, 44, 46, 48, 50, or more, optionally in combination with one or more whole nanocage monomers, self-assemble to form a nanocage.

In an aspect, the nanocage monomer is selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof.

In an aspect, the nanocage monomer is apoferritin.

In an aspect, the first and second nanocage monomer subunits interchangeably comprise the “N” and “C” regions of apoferritin.

In an aspect, the “N” region of apoferritin comprises or consists of a sequence at least 70% (such as at least 75%, 80%, 85%, 90%, 95%, 96%, 97%, 98%, 99%, or 100%) identical to:

MSSQIRQNYSTDVEAAVNSLVNLYLQASYTYLSLGFYFDRDDVALEGVS
HFFRELAEEKREGYERLLKMQNQRGGRALFQDIKKPAEDEW.

In an aspect, the “C” region of apoferritin comprises or consists of a sequence at least 70% (such as at least 75%, 80%, 85%, 90%, 95%, 96%, 97%, 98%, 99%, or 100%) identical to:

GKTPDAMKAAMALEKKLNQALLDLHALGSARTDPHLCDFLETHFLDEEV
KLIKKMGDHLTNLHRLGGPEAGLGEYLFERLTLRHD.

In an aspect, the fusion protein further comprising a linker between the nanocage monomer subunit and the bioactive moiety.

In an aspect, the linker is flexible or rigid and comprises from about 1 to about 30 amino acid residues, such as from about 8 to about 16 amino acid residues.

In an aspect, the linker comprises a GGS repeat, such as 1, 2, 3, 4, or more GGS repeats.

In an aspect, the linker comprises or consists of a sequence at least 70% (such as at least 75%, 80%, 85%, 90%, 95%, 96%, 97%, 98%, 99%, or 100%) identical to:

ASTASSASSGGGGGGSGGSGGSGGS

In an aspect, the fusion protein further comprises a C-terminal linker.

In an aspect, the C-terminal linker comprises or consists of a sequence at least 70% (such as at least 75%, 80%, 85%, 90%, 95%, 96%, 97%, 98%, 99%, or 100%) identical to:

GGSGGSGGSGGSGGGASGGS.

In accordance with an aspect, there is provided a pair of the fusion proteins described herein, wherein the pair self-assembles to form a nanocage monomer, wherein the first and second nanocage monomer subunits are fused to different bioactive moieties.

In accordance with an aspect, there is provided a nanocage comprising at least one fusion protein described herein and at least one second nanocage monomer subunit that self-assembles with the fusion protein to form a nanocage monomer.

In accordance with an aspect, there is provided a nanocage comprising at least one pair described herein.

In an aspect, each nanocage monomer comprises the fusion protein or the pair described herein.

In an aspect, from about 20% to about 80% of the nanocage monomers comprise the fusion protein or the pair described herein.

In an aspect, the fusion protein comprises at least 2, 3, 4, 5, 6, 7, 8, 9, or 10 different bioactive moieties.

In an aspect, the nanocage comprises at least one whole nanocage monomer, optionally fused to a bioactive moiety that may be the same or different from the bioactive moiety described herein.

In an aspect, the nanocage is multivalent and/or multispecific.

In an aspect, the nanocage comprises a first, second, and third fusion protein described herein, and at least one whole nanocage monomer, optionally fused to a bioactive moiety, wherein the bioactive moieties of the first, second, and third fusion proteins and of the whole nanocage monomer are all different from one another.

In an aspect, the first, second, and third fusion proteins each comprise an antibody or fragment thereof fused to N- or C-ferritin, wherein at least one of the first, second, and third fusion proteins is fused to N-ferritin and at least one of the first, second, and third fusion proteins is fused to C-ferritin.

In an aspect, the antibody or fragment thereof of the first fusion protein is an Fc fragment; wherein the second and third fusion proteins each comprise an antibody or fragment thereof specific for a different antigen of a virus such as HIV or wherein one of the second and third fusion proteins comprises an antibody or fragment thereof specific for an antigen of a virus such as HIV and the third fusion protein comprises an antibody or fragment thereof specific for a different antigen, such as the CD4 receptor; and wherein the whole nanocage monomer is fused to a bioactive moiety that is specific for another different antigen, optionally of the same virus such as HIV.

In an aspect, the Fc fragment comprises one or more mutations, such as LS, YTE, LALA, and/or LALAP, that modulate the half-life of the fusion protein from, for example, minutes or hours to several days, weeks, or months.

In an aspect, the antibody or fragment thereof of the second fusion protein is N49P7 or iMab A12P; wherein the antibody or fragment thereof of the third fusion protein is 10E8v4.

In an aspect, the nanocage comprises or consists of the following four fusion proteins:

a. PGDM1400 (optionally scPGDM1400) fused to full length ferritin;

b. Fc (optionally scFc) fused to N-ferritin;

c. N49P7 or iMab A12P (optionally scN49P7 or sciMab A12P) fused to C-ferritin; and

d. 10E8v4 (optionally sc10E8v4) fused to C-ferritin.

In an aspect, the nanocage comprises a 4:2:1:1: ratio of a:b:c:d.

In an aspect, the nanocage comprises or consists of sequences at least 70% (such as at least 75%, 80%, 85%, 90%, 95%, 96%, 97%, 98%, 99%, or 100%) identical to one or more of the following sequences, where ferritin subunits are in bold, linkers are underlined, light chains are italicized, and heavy chains are in lowercase:

a. PGDM1400-hFerr:
DFVLTQSPHSLSVTPGESASISCKSSHSLIHGDRNNYLAWYVQKPGRSP
QLLIYLASSRASGVPDRFSGSGSDKDFTLKISRVETEDVGTYYCMQGRE
SPWTFGQGTKVDIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGECGGSSGSGSGSTGTSSSGTGTSAGTTG
TSASTSGSGSGGGGGSGGGGSAGGTATAGASSGSGSSGSSSSGGTGqaq
lvqsgpevrkpgtsvkvsckapgntlktydlhwvrsvpgqglqwmgwis
hegdkkviverfkakvtidwdrstntaylqlsgltsgdtavyycakgsk
hrlrdyalydddgalnwavdvdylsnlefwgqgtavttvssastkgpsv
fplapsskstsggtaalgclvkdyfpepvtvswnsgaltsgvhtfpavl
qssglyslssvvtvpssslgtqtyivnvnhkpsntkvdkkvepkscdsr
ASTASSASSGGGGGSGGSGGSGGSMSSQIRQNYSTDVEAAVNSLVNLYL
QASYTYLSLGFYFDRDDVALEGVSHFFRELAEEKREGYERLLKMQNQRG
GRALFQDIKKPAEDEWGKTPDAMKAAMALEKKLNQALLDLHALGSARTD
PHLCDFLETHFLDEEVKLIKKMGDHLTNLHRLGGPEAGLGEYLFERLTL
RHDGGSGGSGGSGGSGGGASGGS;
b. Fc-N-hFerr LS
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVLHEALHSHYTQKSLSLSPGKGGSSGSGSGSTGTSSSGT
GTSAGTTGTSASTSGSGSGGGGGSGGGGSAGGTATAGASSGSGSSGSSS
SGGTGdkthtcppcpapellggpsvflfppkpkdtlmisrtpevtcvvv
dvshedpevkfnwyvdgvevhnaktkpreeqynstyrvvsvltvlhqdw
lngkeykckvsnkalpapiektiskakgqprepqvytlppsreemtknq
vsltclvkgfypsdiavewesngqpennykttppvdlsdgsfflysklt
vdksrwqqgnvfscsvlhealhshytqkslslspgksrASTASSASSGG
GGGGSGGSGGSGGSMSSQIRQNYSTDVEAAVNSLVNLYLQASYTYLSLG
FYFDRDDVALESGVSHFFRELAEEKREGYERLLKMQNQRGGRALFQDIK
KPAEDEW;
c1. N49P7-C-hFerr
QSALTQPRSVSASPGQSVTISCTGTHNLVSWCQHQPGRAPKLLIYDFNK
RPSGVPDRFSGSGSGGTASLTITGLQDDDDAEYFCWAYEAFGGGTKLTV
LGQPKAAPSVTLFPPSSEELQANKATLVCLISDFYPGAVTVAWKADSSP
VKAGVETTTPSKQSNNKYAASSYLSLTPEQWKSHRSYSCQVTHEGSTVE
KTVAPTECGGSSGSGSGSTGTSSSGTGTSAGTTGTSASTSGSGSGGGGG
SGGGGSAGGTATAGASSGSGSSGSSSSGGTGadlvqsgavvkkpgdsvr
isceaqgyrfpdyiihwirrapgqgpewmgwmnpmggqvnipwkfqgrv
smtrdtsietafldlrglksddtavyycvrdrsngsgkrfessnwfldl
wgrgtavtiqsastkgpsvfplapsskstsggtaalgclvkdyfpepvt
vswnsgaltsgvhtfpavlqssglyslssvvtvpssslgtqtyicnvnh
kpsntkvdkkvepkscdsrASTASSASSGGGGGGSGGSGGSGGSGKTPD
AMKAAMALEKKLNQALLDLHALGSARTDPHLCDFLETHFLDEEVKLIKK
MGDHLTNLHRLGGPEAGLGEYLFERLTLRHDGGSGGSGGSGGSGGGASG
GS;
c2. Ibalizumab-A12P-C-hFerr
DIVMTQSPDSLPVSLGERVTMNCKSSQSLLYSTNQKNYLAWYQQKPGQS
PKLLIYWASTRESGVPDRFSGSGSGTDFTLTISSVQAEDVAVYYCQQYY
SYRTFGGGTKLEIKRTVAAPSVFIFPPSDEQLKSGTASSVVCLLNNFYP
REAKVQWKVDNALQSGNSQESVTEQDSKDSTYSLSSTLTLSKADYEKHK
VYACEVTHQGLSSPVTKSFNRGECGGSSGSGSGSTGTSSSGTGTSAGTT
GTSASTSGSGSGGGGGSGGGGSAGGTATAGASSGSGSSGSSSSGGTGqv
qlqqsgpevvkpgasvkmsckasgytftsyvihwvrqkpgqgldwigyi
npyndgtdydekfkgkatltsdtststaymelsslrsedtavyycarek
dnyatgawfaywgqgtlvtvssastkgpsvfplapcsrstsestaalgc
lvkdyfpepvtvswnsgaltsgvhtfpavlqssglyslssvvtvpsssl
gtktytcnvdhkpsntkvdkrveskygsrASTASSASSGGGGGGSGGSG
GSGGSGKTPDAMKAAMALEKKLNQALLDLHALGSARTDPHLCDFLETHF
LDEEVKLIKKMGDHLTNLHRLGGPEAGLGEYLFERLTLRHDGGSGGSGG
SGGSGGGASGGS;
or
d. 10E8.v4-C-hFerr
SELTQDPAVSVALKQTVTITCRGDSLRSHYASWYQKKPGQAPVLLFYGK
NNRPSGIPDRFSGSASGNRASLTITGAQAEDEADYYCSSRDKSGSRLSV
FGGGTKLTVLSQPKAAPSVTLFPPSSEELQANKATLVCLISDFYPGAVT
VAWKADSSPVKAGVETTTPSKQSNNKYAASSYLSLTPEQWKSHRSYSCQ
VTHEGSTVEKTVAPTECGGSSGSGSGSTGTSSSGTGTSAGTTGTSASTS
GSGSGGGGGSGGGGSAGGTATAGASSGSGSSGSSSSGGTGevrlvesgg
glvkpggslrlscsasgfdfdnawmtwvrqppgklgewvgritgpgegw
svdyaesvkgrftisrdntkntlylemnnvrtedtgyyfcartgkyydf
wsgyppgeeyfqdwgqgtlvivssastkgpsvfplalsskstsggtaal
gclvkdyfpepvtvswnsgaltsgvhtfpavlqssglyslssvvtvpss
slgtqtyicnvnhkpsntkvdkkvepkscsrASTASSASSGGGGGGSGG
SGGSGGSGKTPDAMKAAMALEKKLNQALLDLHALGSARTDPHLCDFLET
HFLDEEVKLIKKMGDHLTNLHRLGGPEAGLGEYLFERLTLRHDGGSGGS
GGSGGSGGGASGGS.

In an aspect, the nanocage is carrying a cargo molecule, such as a pharmaceutical agent, a diagnostic agent, and/or an imaging agent.

In an aspect, the cargo molecule is not fused to the fusion protein and is contained in the nanocage internally.

In an aspect, the cargo molecule is a protein and is fused to the fusion protein such that the cargo molecule is contained in the nanocage internally.

In an aspect, the cargo molecule is a fluorescent protein, such as GFP, EGFP, Ametrine, and/or a flavin-based fluorescent protein, such as a LOV-protein, such as iLOV.

In an aspect, the cargo molecule is contained internally to provide T-cell epitopes, but optionally not B-cell epitopes.

In an aspect, the cargo molecule is fused to the fusion protein and contained internally to provide T-cell epitopes, but optionally not B-cell epitopes.

In an aspect, the cargo molecule is a small molecule, radioisotope, or magnetic particle.

In an aspect, the nanocage further comprises an antigen on the surface.

In an aspect, the antigen is expressed as a fusion protein with a nanocage monomer.

In accordance with an aspect, there is provided a vaccine comprising the nanocage described herein.

In accordance with an aspect, there is provided a therapeutic or prophylactic composition comprising the nanocage described herein.

In accordance with an aspect, there is provided a nucleic acid molecule encoding the fusion protein or the pair described herein.

In accordance with an aspect, there is provided a vector comprising the nucleic acid molecule described herein.

In accordance with an aspect, there is provided a host cell comprising the vector described herein and producing the fusion protein or the pair described herein.

In accordance with an aspect, there is provided a method of immunizing a subject, the method comprising administering the nanocage or the vaccine described herein.

In accordance with an aspect, there is provided a method for treating and/or preventing a disease or condition, the method comprising administering the nanocage or the vaccine described herein.

In an aspect, the disease or condition is cancer, an infectious disease such as HIV, malaria, influenza, RSV, rotavirus, or an autoimmune disease.

In accordance with an aspect, there is provided a method for diagnostic imaging, the method comprising administering the nanocage described herein to a subject, tissue, or sample, wherein the nanocage comprises a diagnostic label, such as a fluorescent protein or magnetic imaging moiety, and imaging the subject, tissue, or sample.

In accordance with an aspect, there is provided a use of the nanocage or the vaccine described herein for immunizing a subject.

In accordance with an aspect, there is provided a use of the nanocage or the vaccine described herein for treating and/or preventing a disease or condition.

In an aspect, the disease or condition is cancer, an infectious disease such as HIV, malaria, influenza, RSV, rotavirus, or an autoimmune disease.

In accordance with an aspect, there is provided a use of the nanocage described herein for diagnostic imaging of a subject, tissue, or sample, wherein the nanocage comprises a diagnostic label, such as a fluorescent protein or magnetic imaging moiety, and imaging the subject, tissue, or sample.

In accordance with an aspect, there is provided a use of the fusion protein, the pair, or the nanocage described herein as a research tool, such as in FACS or in an ELISA.

In accordance with an aspect, there is provided the nanocage or the vaccine described herein for use in immunizing a subject.

In accordance with an aspect, there is provided the nanocage or the vaccine described herein for use in treating and/or preventing a disease or condition.

In an aspect, the disease or condition is cancer, an infectious disease such as HIV, malaria, influenza, RSV, rotavirus, or an autoimmune disease.

In accordance with an aspect, there is provided the nanocage described herein for use in diagnostic imaging of a subject, tissue, or sample, wherein the nanocage comprises a diagnostic label, such as a fluorescent protein or magnetic imaging moiety, and imaging the subject, tissue, or sample.

In accordance with an aspect, there is provided the fusion protein, the pair, or the nanocage described herein for use as a research tool, such as in FACS or in an ELISA.

In accordance with an aspect, there is provided a nanocage comprising a plurality of fusion proteins,

wherein each fusion protein comprises a ferritin light chain and an Fab fragment,

wherein each Fab fragment is capable of specifically binding to an antigen,

wherein each Fab fragment decorates the exterior surface of the nanocage, and

wherein the plurality comprises at least 12 fusion proteins.

In an aspect, the plurality comprises at least 19 fusion proteins.

In an aspect, the plurality comprises at least 24 fusion proteins.

In an aspect, the plurality is 24 fusion proteins.

In an aspect, the Fab fragments of the plurality of fusion proteins are capable of specifically binding to the same antigen.

In an aspect, the nanocage does not include any ferritin heavy chains.

In an aspect, the Fab fragments are Fab fragments of a neutralizing antibody.

In an aspect, the antigen is associated with an infectious agent.

In an aspect, the infectious agent is a virus.

In an aspect, the virus is a human immunodeficiency virus (HIV).

In an aspect, the nanocage is capable of neutralizing the infectious agent with a neutralizing activity of at least 100-fold, 150-fold, 200-fold, 250-fold, 300-fold, 350-fold, 400-fold, 450-fold, or 500-fold greater relative to a control.

In an aspect, the control comprises a full length version of the neutralizing antibody.

In an aspect, the neutralizing antibody is an IgG antibody.

In accordance with an aspect, there is provided a nanocage comprising a plurality of first fusion proteins and a plurality of second fusion proteins,

wherein each first fusion protein comprises a nanocage monomer or a subunit thereof and an Fab fragment capable of specifically binding to an antigen, and

wherein each second fusion protein comprises a nanocage monomer or a subunit thereof and an Fc fragment.

In an aspect, the nanocage monomer is selected from ferritin, apoferritin, encapsulin, sulfur oxygen reductase (SOR), lumazine synthase, pyruvate dehydrogenase, carboxysome, vault proteins, GroEL, heat shock protein, E2P, MS2 coat protein, fragments thereof, and variants thereof.

In an aspect, the nanocage monomer is apoferritin or ferritin.

In an aspect, the nanocage monomer is a ferritin light chain.

In an aspect, the nanocage monomer does not include any ferritin heavy chains.

In accordance with an aspect, there is provided a nanocage comprising a plurality of first fusion proteins and a plurality of second fusion proteins, wherein

(a) (i) the first fusion protein comprises a ferritin light chain and an Fab fragment capable of specifically binding to a first antigen, and
(ii) the second fusion protein comprises a ferritin light chain and an Fab fragment capable of specifically binding to a second antigen, or

(b) (i) the first fusion protein comprises N-ferritin and an Fab fragment capable of specifically binding to a first antigen, and
(ii) the second fusion protein comprises C-ferritin and an Fab fragment capable of specifically binding to a second antigen,

wherein, within each fusion protein, the Fab fragment is fused to the N-terminus of the ferritin light chain, the N-ferritin, or the C-ferritin, and

wherein the first antigen is distinct from the second antigen.

In accordance with an aspect, there is provided a nanocage comprising a plurality of first fusion proteins, a plurality of second fusion proteins, and a plurality of third fusion proteins, wherein

(a) the first fusion protein comprises a ferritin light chain and an Fab fragment capable of specifically binding a first antigen,

(b) the second fusion protein comprises a C-ferritin and an Fab fragment capable of specifically binding a second antigen, and

(c) the third fusion protein comprises an N-ferritin and an Fc fragment,

wherein, within each fusion protein the Fab or Fc fragment is fused to the N-terminus of the ferritin light chain, the C-ferritin, or the N-ferritin, and

wherein the first antigen is distinct from the second antigen.

In an aspect, the nanocage further comprises a plurality of fourth fusion proteins, wherein, the fourth fusion protein comprises a C-ferritin and an Fab fragment capable of specifically binding a third antigen, wherein the third antigen is distinct from the first and second antigens.

In an aspect, the Fab fragments are Fab fragments of a neutralizing antibody.

In an aspect, the first and second antigens are each associated with an infectious agent.

In an aspect, the first and second antigens are associated with the same infectious agent.

In an aspect, the infectious agent is a virus.

In an aspect, the virus is a human immunodeficiency virus (HIV).

In an aspect, the first and second antigens are each associated with a virus,

wherein the nanocage is capable of neutralizing 100% of pseudoviruses in a panel of pseudoviruses, and

wherein the panel of pseudoviruses comprises, for each Fab fragment within the nanocage capable of specifically binding to an antigen associated with the virus, at least one pseudovirus resistant to a neutralizing antibody corresponding to that Fab fragment.

In an aspect, the panel of pseudoviruses comprises at least 10, at least 11, at least 12, at least 13, or at least 14 pseudoviruses.

In an aspect, the first and second antigens are each associated with a virus,

wherein the nanocage is capable of neutralizing a panel of pseudoviruses with an IC₅₀ of less than 1 nM, less than 500 pM, less than 250 pM, less than 100 pM, less than 50 pM, less than 10 pM, or less than 5 pM, and

wherein the panel of pseudoviruses comprises, for each Fab fragment within the nanocage capable of specifically binding to an antigen associated with the virus, at least one pseudovirus resistant to a neutralizing antibody corresponding to that Fab fragment.

In an aspect, the first and second antigens are each associated with a virus,

wherein the nanocage is capable of neutralizing a panel of pseudoviruses with an IC₅₀ (molar concentration) of at least 10-fold, at least 20-fold, at least 30-fold, at least 40-fold, at least 50-fold, at least 60-fold, at least 70-fold, at least 80-fold, at least 90-fold, or at least 100-fold lower than that of one or more controls, and

wherein the panel of pseudoviruses comprises, for each Fab fragment within the nanocage capable of specifically binding to an antigen associated with the virus, at least one pseudovirus resistant to a neutralizing antibody corresponding to that Fab fragment.

In an aspect, the one or more controls comprise a neutralizing antibody corresponding to a Fab fragment within the nanocage, which Fab fragment is capable of specifically binding to an antigen associated with the virus.

In an aspect, the neutralizing antibody is an IgG antibody.

In an aspect, the one or more controls comprise a cocktail of neutralizing antibodies, wherein the cocktail comprises, for each Fab fragment within the nanocage capable of specifically binding to an antigen associated with the virus, a neutralizing antibody corresponding to that Fab fragment.

In an aspect, the neutralizing antibodies are IgG antibodies.

In an aspect, the one or more controls comprise one or more multispecific antibodies, wherein the one or more multispecific antibodies are collectively capable of binding the first and second antigens, and, optionally, the third antigen.

In an aspect, the one or more controls comprise a trispecific antibody capable of specifically binding to the first, second, and third antigens.

In an aspect, the first, second, and third antigens are associated with HIV-1; and wherein:

the Fab fragment of the first fusion protein is a PDGM1400 Fab,

the Fab fragment of the second fusion protein is a 10E8v4 Fab,

the Fc fragment of the third fusion protein is a human IgG1 Fc fragment, and

the Fab fragment of fourth fusion protein is a N49P7 Fab.

In an aspect, the first and second antigens are associated with HIV-1; wherein the third antigen is associated with CD4; and wherein:

the Fab fragment of the first fusion protein is a PDGM1400 Fab,

the Fab fragment of the second fusion protein is a 10E8v4 Fab,

the Fc fragment of the third fusion protein is a human IgG1 Fc fragment, and

the Fab fragment of fourth fusion protein is an iMab Fab.

In accordance with an aspect, there is provided a therapeutic or prophylactic composition comprising the nanocage described herein.

In accordance with an aspect, there is provided a method for treating or preventing a disease or condition, the method comprising administering the nanocage or the composition described herein to a subject in need thereof.

In accordance with an aspect, there is provided a method of making a multispecific self-assembling nanocage, the nanocage characterized by a pre-selected ratio of different specificities, the method comprising the step of:

co-transfecting a host cell with one or more expression plasmids comprising a plurality of polynucleotides, each polynucleotide encoding a fusion protein,
wherein each fusion protein comprises (i) a nanocage monomer or a subunit thereof and (ii) an antibody or antibody fragment of a given specificity,
wherein the step of co-transfecting comprises co-transfecting the polynucleotides in a ratio based on the pre-selected ratio;

obtaining polypeptides produced by the host cell; and

purifying the polypeptides by affinity selection for all the different specificities to be present in an assembled nanocage.

In an aspect, the plurality of polynucleotides comprises at least one polynucleotide encoding a first fusion protein and at least one polynucleotide encoding a second fusion protein,

wherein the first fusion protein comprises a first nanocage monomer subunit, and

wherein the second fusion protein comprises a second nanocage monomer subunit capable of self-assembling with the first nanocage monomer subunit.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1. Schematic for the self-assembly of the MULTi-specific, multi-Affinity antiBODY (multabody) platform. The single-chain Fab (light chain (LC) and heavy chain (HC) in light and dark pink, respectively) and single-chain Fc region (green) are connected to the N-terminus of the light chain of human apoferritin (grey) through a GGS-like flexible linker (dark). 24 subunits of apoferritin self-assemble in a 12 nm spherical core surrounded by spatially dispersed antibody fragments.

FIG. 2. Characterization of HIV-1 multabodies with different valency. (a) Schematic representation of different Fab densities displayed on human apoferritin. Co-transfection of scFab-human apoferritin-encoding plasmids together with different ratios of unconjugated apoferritin resulted in scFab valency of 5 (dark yellow), 12 (black), 19 (blue) and 24 (red), as confirmed by earlier elution volumes and less unconjugated apoferritin in size exclusion chromatography and SDS-PAGE, respectively. Negative stain electron micrographs of the samples with the lowest (20%) and highest (100%) valency are shown. (b) Avidity effect for neutralization of five bNAbs against a five-PsV panel (PVO.04, JRCSF, BG505 T332N, THRO4156.18 and t278-50). IC50-fold increase analysis was omitted in the following cases: N49P7-t278-50, VRC01-T278-50 and 10-1074-THR04156.18 due to the neutralization resistance. Potency fold increase was calculated as the parental IgG IC₅₀ (nM) divided by the multabody IC₅₀ (nM).

FIG. 3. Design, assembly and biophysical characterization of 32-N and 32-I multabodies. (a) Schematic for the human apoferritin split design that favors hetero-dimerization of scFab-human apoferritin subunits. The resulting two halves termed N-ferritin and C-ferritin span residues 1 to 95 and 95 to 175, respectively. scFc was linked at the N-terminus of the N-ferritin half while N49P7, iMab (also referred to herein as ibalizumab) and 10E8v4 were connected to the N-terminus of the C-ferritin half. Hetero-dimerization of the split halves drives the self-assembly of different antibody fragments, resulting in the formation of a single human apoferritin subunit with two cargos. Further combination of these constructs with PGDM1400 scFab linked to a full human apoferritin subunit causes the assembly of nanoparticles displaying a mixture of 32 scFab/scFc on the surface of the multabody. Negative stain electron micrographs, a model representation of the scFab/scFc 32-N/32-1 design, and the specific composition of the 32-N and 32-I multabodies is shown. Based on the necessary hetero-oligomerization to drive self-assembly, purification of multabodies with the four components can be achieved by a two-step purification: protein A (Fc binding) and protein L (PGDM1400 binding). (b) Size exclusion chromatography in-line with multi-angle light scattering of 24-mer PGDM1400 multabodies (black), 32-N multabodies (dark magenta) and 32-1 multabodies (blue). The molar mass of each elution peak (lines under UV absorbance) shows the sample is monodisperse, and that the 32-N/32-I multabodies are significantly bigger than the 24-mer format due to additional antibody fragments in this design. (c) Comparison on the Tₘ and T_{agg} temperatures of 32-N/32-I multabodies, 12-mer multabodies, parental IgGs and the N6/PGDM1400x10E8v4 trispecific antibody. (d) Concentration-response curves for binding of 32-N and 32-1 multabodies to multiple epitopes. PGDM1400, N49P7 and 10E8 binding sites are colored in red, blue and pink, respectively in the surface representation of the HIV Env (in grey). Binding of iMab was assessed using soluble CD4 while functional binding of Fc to human FcRn was tested by measuring binding at pH 7.5 and pH 5.6. The BG505 SOSIP.664_D368R trimer and 93TH057 gp120 monomer were selected as epitope-specific ligands for PGDM1400 and N49P7, respectively.

FIG. 4. Effect of the split design on the biophysical and functional properties of the multabodies. Comparison of 12-mer multabodies composed of 6 copies of PGDM1400 and 6 copies of Fc multimerized with a full apoferritin subunit (left panels) or half ferritin (Fc attached to the N-Ferritin half and PGDM1400 linked to the C-Ferritin half, right panels). a) Biolayer interferometry (BLI) concentration-response curves for binding of BG505.664 to the 12-mer multabodies loaded onto anti-hlgG Fc capture (AHC) biosensors. b) Barycentric mean fluorescence (BCM) and static light scattering (SLS) at 266 nm versus temperature plots. Tₘ and T_{agg} are indicated with yellow lines. c) Neutralization assay against the BG505 T332N PsV.

FIG. 5. Multabody affinity-purification scheme. Protein A and Protein L sequential affinity purification. Binding to Protein A enriches for multabodies with Fc (green) while Protein L enriches for multabodies with the kappa chain Fab PGDM1400 (blue). Alanine-to-proline point mutation at position 12 of the kappa chain of iMab was introduced to disrupt binding to Protein L⁷⁵. Complementation of the two halves of human apoferritin ensures the presence of N49P7/iMab (orange) and 10E8 scFabs (pink) (fused to C-ferritin) during the protein A purification step. Gel filtration is performed to separate any aggregated material.

FIG. 6. Batch-to-batch variation of the 32-N multabody is minimal. a) SEC chromatograms. b) BCM and SLS at 266 nm versus temperature plots. Thermal transition temperatures (Tₘ and T_{agg}) are indicated with yellow lines. c) Concentration-response curves for binding of N49P7 within 32-N to 93TH057 gp120. d) Neutralization profiles of 32-N against a panel of four PsV selected to include one PsV resistant to each of the Fabs in the multabody.

FIG. 7. Thermostability analysis. BCM (top panels) and SLS (bottom panels) at 266 nm versus temperature plots for 32-N and 32-I multabodies, their individual 12-mer multabodies, parental IgGs and the N6/PGDM1400x10E8 trispecific antibody. Thermal transition temperatures (Tₘ and T_{agg}) are indicated with yellow lines.

FIG. 8. Binding profile of bNAbs PGDM1400, 10E8v4, N49P7 and iMab. BLI response curves of IgG binding to 93TH057 gp120, BG505 SOSIP.664_D368R, MPER-mVenus and CD4 immobilized onto Ni-NTA biosensors.

FIG. 9. Neutralization properties of 32-N and 32-I multabodies against a 14-pseudovirus panel. Breadth and median 1050 values (μg/mL) of multabodies (red diamonds), parental bNAbs (black circles), IgG combinations (back triangles) and the N6/PGDM1400x10E8v4 trispecific antibody (black square). The IgG cocktails contained each of the parental antibodies in the same relative amount as in the multabody samples (i.e. 66% PGDM1400, 17% N49P7/iMab and 17% 10E8v4). The 14-PsV panel was selected based on susceptibility and resistance to the parental IgGs.

FIG. 10. Neutralization properties of 32-N and 32-I multabodies against a 14-pseudovirus panel. Breadth and median 1050 values (nM) of multabodies (red diamonds), parental bNAbs (black circles), IgG combinations (66% PGDM1400, 17% N497/iMab and 17% 10E8v4, black triangles) and the N6/PGDM1400x10E8v4 trispecific antibody (black square).

FIG. 11. Immunogenicity and exposure of a multabody in mice. Five male C57BL/6 mice per group were used to assess anti-drug antibody and multabody circulation in blood after subcutaneous administration of 5 mg/kg of a mouse surrogate multabody, and a Fc-modified multabody (LALAP mutation to disrupt Fc receptor binding). Reference samples HpFerritin malaria PfCSP peptide and parental mouse IgG1 and IgG2a isotypes were used for comparison of immunogenicity and exposure, respectively.

### DETAILED DESCRIPTION OF CERTAIN ASPECTS

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only, and is not intended to be limiting. Many patent applications, patents, and publications are referred to herein to assist in understanding the aspects described. Each of these references are incorporated herein by reference in their entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation. For example, in some aspects the nanocages and/or fusion proteins described herein may exclude a ferritin heavy chain and/or may exclude an iron-binding component.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

The terms “protein nanoparticle,” “nanocage,” and “multabody” are used interchangeably herein and refer to a multi-subunit, protein-based polyhedron shaped structure. The subunits or nanocage monomers are each composed of proteins or polypeptides (for example a glycosylated polypeptide), and, optionally of single or multiple features of the following: nucleic acids, prosthetic groups, organic and inorganic compounds. Non-limiting examples of protein nanoparticles include ferritin nanoparticles (see, e.g., Zhang, Y. Int. J. Mol. Sci., 12:5406-5421, 2011, incorporated by reference herein), encapsulin nanoparticles (see, e.g., Sutter et al., Nature Struct, and Mol. Biol., 15:939-947, 2008, incorporated by reference herein), Sulfur Oxygenase Reductase (SOR) nanoparticles (see, e.g., Urich et al., Science, 311:996-1000, 2006, incorporated by reference herein), lumazine synthase nanoparticles (see, e.g., Zhang et al., J. Mol. Biol., 306: 1099-1114, 2001) or pyruvate dehydrogenase nanoparticles (see, e.g., Izard et al., PNAS 96: 1240-1245, 1999, incorporated by reference herein). Ferritin, apoferritin, encapsulin, SOR, lumazine synthase, and pyruvate dehydrogenase are monomeric proteins that self-assemble into a globular protein complexes that in some cases consists of 24, 60, 24, 60, and 60 protein subunits, respectively. Ferritin and apoferritin are generally referred to interchangeably herein and are understood to both be suitable for use in the fusion proteins, nanocages, and methods described herein. Carboxysome, vault proteins, GroEL, heat shock protein, E2P and MS2 coat protein also produce nanocages are contemplated for use herein. In addition, fully or partially synthetic self-assembling monomers are also contemplated for use herein.

It will be understood that each nanocage monomer may be divided into two or more subunits that will self-assemble into a functional nanocage monomer. For example, ferritin or apoferritin may be divided into an N- and C-subunit, e.g., an N- and C-subunit obtained by dividing full-length ferritin substantially in half, so that each subunit may be separately bound to a different bioactive moiety for subsequent self-assembly into a nanocage monomer and then a nanocage. By “functional nanocage monomer” it is intended that the nanocage monomer is capable of self-assembly with other such monomers into a nanocage as described herein.

The terms “ferritin” and “apoferritin” are used interchangeably herein and generally refer to a polypeptide (e.g., a ferritin chain) that is capable of assembling into a ferritin complex which typically comprises 24 protein subunits. It will be understood that the ferritin can be from any species. Typically, the ferritin is a human ferritin. In some embodiments, the ferritin is a wild-type ferritin. For example, the ferritin may be a wild-type human ferritin. In some embodiments, a ferritin light chain is used as a nanocage monomer, and/or a subunit of a ferritin light chain is used as a nanocage monomer subunit. In some embodiments, assembled nanocages do not include any ferritin heavy chains or other ferritin components capable of binding to iron.

The term “multispecific,” as used herein, refers to the characteristic of having at least two binding sites at which at least two different binding partners, e.g., an antigen or receptor (e.g., Fc receptor), can bind. For example, a nanocage that comprises at least two Fab fragments, wherein each of the two Fab fragments binds to a different antigen, is “multispecific.” As an additional example, a nanocage that comprises an Fc fragment (which is capable of binding to an Fc receptor) and an Fab fragment (which is capable of binding to an antigen) is “multispecific.”

The term “multivalent,” as used herein, refers to the characteristic of having at least two binding sites at which a binding partner, e.g., an antigen or receptor (e.g., Fc receptor), can bind. The binding partners that can bind to the at least two binding sites may be the same or different.

A “vaccine” is a pharmaceutical composition that induces a prophylactic or therapeutic immune response in a subject. In some cases, the immune response is a protective immune response. Typically, a vaccine induces an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In one specific, non-limiting example, a vaccine induces an immune response that reduces the severity of the symptoms associated with malaria infection and/or decreases the parasite load compared to a control. In another non-limiting example, a vaccine induces an immune response that reduces and/or prevents malaria or HIV infection compared to a control.

The term “antibody”, also referred to in the art as “immunoglobulin” (Ig), used herein refers to a protein constructed from paired heavy and light polypeptide chains; various Ig isotypes exist, including IgA, IgD, IgE, IgG, such as IgG₁, IgG₂, IgG₃, and IgG₄, and IgM. It will be understood that the antibody may be from any species, including human, mouse, rat, monkey, llama, or shark. When an antibody is correctly folded, each chain folds into a number of distinct globular domains joined by more linear polypeptide sequences. For example, the immunoglobulin light chain folds into a variable (V_{L}) and a constant (CL) domain, while the heavy chain folds into a variable (V_{H}) and three constant (C_{H}, C_{H2}, C_{H3}) domains. Interaction of the heavy and light chain variable domains (V_{H} and V_{L}) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important immunological events. The variable region of an antibody contains the antigen binding determinants of the molecule, and thus determines the specificity of an antibody for its target antigen. The majority of sequence variability occurs in six hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape and chemistry of the surface they present to the antigen.

An “antibody fragment” as referred to herein may include any suitable antigen-binding antibody fragment known in the art. The antibody fragment may be a naturally-occurring antibody fragment, or may be obtained by manipulation of a naturally-occurring antibody or by using recombinant methods. For example, an antibody fragment may include, but is not limited to a Fv, single-chain Fv (scFv; a molecule consisting of V_{L} and V_{H} connected with a peptide linker), Fc, single-chain Fc, Fab, single-chain Fab, F(ab′)₂, single domain antibody (sdAb; a fragment composed of a single V_{L} or V_{H}), and multivalent presentations of any of these.

By the term “synthetic antibody” as used herein, is meant an antibody which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “epitope” refers to an antigenic determinant. An epitope is the particular chemical groups or peptide sequences on a molecule that are antigenic, that is, that elicit a specific immune response. An antibody specifically binds a particular antigenic epitope, e.g., on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, about 11, or about 8 to about 12 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996).

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the aspects described herein include, but are not limited to, the use of partial nucleotide sequences of more than one gene and that these nucleotide sequences could be arranged in various combinations to elicit the desired immune response. Moreover, a skilled artisan will understand that an antigen need not be encoded by a “gene” at all. It is readily apparent that an antigen can be synthesized or can be derived from a biological sample. Such a biological sample can include, but is not limited to a tissue sample, a cell, or a biological fluid.

Thus, the compositions described herein may be suitable for protection or treatment of vertebrate subjects against a variety of disease states such as, for example, viral, bacterial, fungal or parasitic infections, cancer, and autoimmune disorders. It is to be recognized that these specific disease states have been referred to by way of example only and are not intended to be limiting.

Suitable antigens useful in combination with the compositions described herein include any antigen as defined herein. Antigens are commercially available or one of skill in the art is capable of producing them. The antigen can be either a modified-live or killed microorganism, or a natural product purified from a microorganism or other cell including, but not limited to, tumor cell, a synthetic product, a genetically engineered protein, peptide, polysaccharide or similar product, or an allergen. The antigenic moiety can also be a subunit of a protein, peptide, polysaccharide or similar product. The antigen may also be a genetic antigen, i.e., DNA or RNA that engenders an immune response.

Representative of the antigens that can be used include, but are not limited to, natural, recombinant or synthetic products derived from viruses, bacteria, fungi, parasites and other infectious agents in addition to autoimmune diseases, hormones, or tumor antigens which might be used in prophylactic or therapeutic vaccines and allergens. In one embodiment, the antigen comprises virus-like particles (VLPs) from various viruses such as influenza, HIV, RSV, Newcastle disease virus (NDV) etc. See PCT/US2006/40862, PCT/US2004/022001, U.S. Ser. No. 11/582,540, U.S. 60/799,343, U.S. 60/817,402, U.S. 60/859,240, all of which are herein incorporated by reference in their entirety. In another embodiment, the antigen comprises chimeric VLPs. “Chimeric VLPs” refer to VLPs that contain proteins, or portions thereof, from at least two different sources (organisms). Usually, one protein is derived from a virus that can drive the formation of VLPs from host cells. Thus, in one embodiment, said chimeric VLP comprises an RSV M protein. In another embodiment, said chimeric VLP comprises a NDV M protein. In another embodiment, said chimeric VLP comprises an influenza virus M protein.

The viral or bacterial products can be components which the organism produced by enzymatic cleavage or can be components of the organism that were produced by recombinant DNA techniques that are well known to those of ordinary skill in the art.

Some specific examples of antigens are antigens derived from viral infections caused by hepatitis viruses A, B, C, D & E3, human immunodeficiency virus (HIV), herpes viruses 1, 2, 6 & 7, cytomegalovirus, varicella zoster, papilloma virus, Epstein Barr virus, para-influenza viruses, adenoviruses, bunya viruses (e.g. hanta virus), coxsakie viruses, picoma viruses, rotaviruses, respiratory syncytial viruses, rhinoviruses, rubella virus, papovavirus, mumps virus, measles virus, polio virus (multiple types), adeno virus (multiple types), parainfluenza virus (multiple types), avian or pandemic influenza (various types), seasonal influenza, shipping fever virus, Western and Eastern equine encephalomyelitis, Japanese B. encephalomyelitis, Russian Spring Summer encephalomyelitis, hog cholera virus, Newcastle disease virus, fowl pox, rabies, feline and canine distemper and the like viruses, slow brain viruses, rous sarcoma virus (RSV), Papovaviridae, Parvoviridae, Picornaviridae, Poxyiridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, Japanese B encephalitis, Junin, Chikungunya, Rift Valley fever, yellow fever, meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia.

The antigens may also be derived from bacterial and fungal infections for example: antigens derived from infections caused by Mycobacteria causing TB and leprosy, pneumocci, aerobic gram negative bacilli, mycoplasma, staphyloccocal infections, streptococcal infections, salmonellae and chlamydiae, B. pertussis, Leptospira pomona, and icterohaemorrhagiae. Specific embodiments comprise S. paratyphi A and B, C. diphtheriae, C. tetani, C. botulinum, C. perfringens, C. feseri and other gas gangrene bacteria, B. anthracis, P. pestis, P. multocida, Neisseria meningitidis, N. gonorrheae, Hemophilus influenzae, Actinomyces (e.g., Norcardia), Acinetobacter, Bacillaceae (e.g., Bacillus anthrasis), Bacteroides (e.g., Bacteroides fragilis), Blastomycosis, Bordetella, Borrelia (e.g., Borrelia burgdorferi), Brucella, Candidia, Campylobacter, Chlamydia, Coccidioides, Corynebacterium (e.g., Corynebacterium diptheriae), Cryptococcus, Dermatocycoses, E. coli (e.g., Enterotoxigenic E. coli and Enterohemorrhagic E. coli), Enterobacter (e.g. Enterobacter aerogenes), Enterobacteriaceae (Klebsiella, Salmonella (e.g., Salmonella typhi, Salmonella enteritidis, Serratia, Yersinia, Shigella), Erysipelothrix, Haemophilus (e.g., Haemophilus influenza type B), Helicobacter, Legionella (e.g., Legionella pneumophila), Leptospira, Listeria (e.g., Listeria monocytogenes), Mycoplasma, Mycobacterium (e.g., Mycobacterium leprae and Mycobacterium tuberculosis), Vibrio (e.g., Vibrio cholerae), Pasteurellacea, Proteus, Pseudomonas (e.g., Pseudomonas aeruginosa), Rickettsiaceae, Spirochetes (e.g., Treponema spp., Leptospira spp., Borrelia spp.), Shigella spp., Meningiococcus, Pneumococcus and Streptococcus (e.g., Streptococcus pneumoniae and Groups A, B, and C Streptococci), Ureaplasmas, Treponema pollidum, and the like; Staphylococcus aureus, Plasmodium sp. (Pl. falciparum, Pl. vivax, etc.), Aspergillus sp., Candida albicans, Pasteurella haemolytica, Corynebacterium diptheriae toxoid, Meningococcal polysaccharide, Bordetella pertusis, Streptococcus pneumoniae (pneumococcus) polysaccharide, Clostridium tetani toxoid, Mycobacterium bovis, killed cells of Salmonella typhi, Cryptococcus neoformans, and Aspergillus.

The antigens may also be derived from parasitic malaria, leishmaniasis, trypanosomiasis, toxoplasmosis, schistosomiasis, filariasis malaria, Amebiasis, Babesiosis, Coccidiosis, Cryptosporidiosis, Dientamoebiasis, Dourine, Ectoparasitic, Giardias, Helminthiasis, Theileriasis, Trichomonas and Sporozoans (e.g., Plasmodium vivax, Plasmodium falciparum, Plasmodium malariae, Plasmodium knowlesi and Plasmodium ovale). These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), malaria, pregnancy complications, and toxoplasmosis.

Tumor-associated antigens suitable for use in compositions described herein include both mutated and non-mutated molecules which may be indicative of single tumor type, shared among several types of tumors, and/or exclusively expressed or overexpressed in tumor cells in comparison with normal cells. In addition to proteins and glycoproteins, tumor-specific patterns of expression of carbohydrates, gangliosides, glycolipids and mucins have also been documented. Exemplary tumor-associated antigens for use in the subject cancer vaccines include protein products of oncogenes, tumor suppressor genes and other genes with mutations or rearrangements unique to tumor cells, reactivated embryonic gene products, oncofetal antigens, tissue-specific (but not tumor-specific) differentiation antigens, growth factor receptors, cell surface carbohydrate residues, foreign viral proteins and a number of other self proteins. Specific embodiments of tumor-associated antigens include, e.g., mutated antigens such as the protein products of the Ras p21 protooncogenes, tumor suppressor p53 and HER-2/neu and BCR-ab1 oncogenes, as well as CDK4, MUM1, Caspase 8, and Beta catenin; overexpressed antigens such as galectin 4, galectin 9, carbonic anhydrase, Aldolase A, PRAME, Her2/neu, ErbB-2 and KSA, oncofetal antigens such as alpha fetoprotein (AFP), human chorionic gonadotropin (hCG); self antigens such as carcinoembryonic antigen (CEA) and melanocyte differentiation antigens such as Mart 1/Melan A, gp100, gp75, Tyrosinase, TRP1 and TRP2; prostate associated antigens such as PSA, PAP, PSMA, PSM-P1 and PSM-P2; reactivated embryonic gene products such as MAGE 1, MAGE 3, MAGE 4, GAGE 1, GAGE 2, BAGE, RAGE, and other cancer testis antigens such as NY-ESO1, SSX2 and SCP1; mucins such as Muc-1 and Muc-2; gangliosides such as GM2, GD2 and GD3, neutral glycolipids and glycoproteins such as Lewis (y) and globo-H; and glycoproteins such as Tn, Thompson-Freidenreich antigen (TF) and sTn. Also included as tumor-associated antigens herein are whole cell and tumor cell lysates as well as immunogenic portions thereof, as well as immunoglobulin idiotypes expressed on monoclonal proliferations of B lymphocytes for use against B cell lymphomas. Tumor-associated antigens and their respective tumor cell targets include, e.g., cytokeratins, particularly cytokeratin 8, 18 and 19, as antigens for carcinoma. Epithelial membrane antigen (EMA), EphA1, EphA2, EphA3, EphA4, EphA5, EphA6, EphA7, EphA8, EphA10, EphB1, EphB2, EphB3, EphB4, EphB6, human embryonic antigen (HEA-125), human milk fat globules, MBr1, MBr8, Ber-EP4, 17-1A, C26 and T16 are also known carcinoma antigens. Desmin and muscle-specific actin are antigens of myogenic sarcomas. Placental alkaline phosphatase, beta-human chorionic gonadotropin, and alpha-fetoprotein are antigens of trophoblastic and germ cell tumors. Prostate specific antigen is an antigen of prostatic carcinomas, carcinoembryonic antigen of colon adenocarcinomas. HMB-45 is an antigen of melanomas. In cervical cancer, useful antigens could be encoded by human papilloma virus. Chromogranin-A and synaptophysin are antigens of neuroendocrine and neuroectodermal tumors. Of particular interest are aggressive tumors that form solid tumor masses having necrotic areas. The lysis of such necrotic cells is a rich source of antigens for antigen-presenting cells, and thus the subject therapy may find advantageous use in conjunction with conventional chemotherapy and/or radiation therapy. The antigens can be derived from any tumor or malignant cell line.

Antigens may also be derived from common allergens that cause allergies. Allergens include organic or inorganic materials derived from a variety of man-made or natural sources such as plant materials, metals, ingredients in cosmetics or detergents, latexes, or the like. Classes of suitable allergens for use in the compositions and methods described herein can include, but are not limited to, pollens, animal dander, grasses, molds, dusts, antibiotics, stinging insect venoms, and a variety of environmental (including chemicals and metals) drug and food allergens. Common tree allergens include pollens from cottonwood, popular, ash, birch, maple, oak, elm, hickory, and pecan trees; common plant allergens include those from rye, ragweed, English plantain, sorrel-dock and pigweed; plant contact allergens include those from poison oak, poison ivy and nettles; common grass allergens include Timothy, Johnson, Bermuda, fescue and bluegrass allergens; common allergens can also be obtained from molds or fungi such as Alternaria, Fusarium, Hormodendrum, Aspergillus, Micropolyspora, Mucor and thermophilic actinomycetes; penicillin and tetracycline are common antibiotic allergens; epidermal allergens can be obtained from house or organic dusts (typically fungal in origin), from insects such as house mites (Dermalphagoides pterosinyssis), or from animal sources such as feathers, and cat and dog dander; common food allergens include milk and cheese (diary), egg, wheat, nut (e.g., peanut), seafood (e.g., shellfish), pea, bean and gluten allergens; common environmental allergens include metals (nickel and gold), chemicals (formaldehyde, trinitrophenol and turpentine), Latex, rubber, fiber (cotton or wool), burlap, hair dye, cosmetic, detergent and perfume allergens; common drug allergens include local anesthetic and salicylate allergens; antibiotic allergens include penicillin and sulfonamide allergens; and common insect allergens include bee, wasp and ant venom, and cockroach calyx allergens. Particularly well characterized allergens include, but are not limited to, the major and cryptic epitopes of the Der pl allergen (Hoyne et al. (1994) Immunology 83, 190-195), bee venom phospholipase A2 (PLA) (Akdis et al. (1996) J. Clin. Invest. 98, 1676-1683), birch pollen allergen Bet v 1 (Bauer et al. (1997) Clin. Exp. Immunol. 107, 536-541), and the multi-epitopic recombinant grass allergen rKBG8.3 (Cao et al. (1997) Immunology 90, 46-51). These and other suitable allergens are commercially available and/or can be readily prepared as extracts following known techniques.

The antigen may be in the form of purified or partially purified antigen and can be derived from any of the above antigens, an antigenic peptide, proteins that are known and available in the art, and others that can identified using conventional techniques. The antigens will typically be in the form in which their toxic or virulent properties have been reduced or destroyed and which when introduced into a suitable, will either induce and immune response against the specific microorganisms, extract, or products of microorganisms used in the preparation of the antigen, or, in the case of allergens, they will aid in alleviating the symptoms of the allergy due to the specific allergen. The antigens can be used either singly or in combination; for example, multiple bacterial antigens, multiple viral antigens, multiple bacterial antigens, multiple parasitic antigens, multiple bacterial, viral toxoids, multiple tumor antigens, multiple allergens or combinations of any of the foregoing products can be combined with adjuvant compositions to create a polyvalent antigenic composition and/or a vaccine. In the compositions described herein, the antigen may be antigen entrapped in, adsorbed to, or in an admixture with the vesicle component of the composition.

In one embodiment, suitable antigens for use with the compositions described herein include antigens which are poorly immunogenic, for example malaria antigens, dengue antigens and HIV antigens, or antigens intended to confer immunity against pandemic diseases, for example influenza antigens. Combinations of any such antigens described herein or known are contemplated for use in the fusion proteins, pairs of fusion proteins, and nanocages described herein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The terms “therapeutically effective amount”, “effective amount” or “sufficient amount” mean a quantity sufficient, when administered to a subject, including a mammal, for example a human, to achieve a desired result, for example an amount effective to cause a protective immune response. Effective amounts of the compounds described herein may vary according to factors such as the immunogen, age, sex, and weight of the subject. Dosage or treatment regimes may be adjusted to provide the optimum therapeutic response, as is understood by a skilled person. For example, administration of a therapeutically effective amount of the fusion proteins described herein is, in aspects, sufficient to increase immunity against a pathogen, such as Plasmodium or HIV. In other aspects, administration of a therapeutically effective amount of the fusion proteins described herein is sufficient to treat a disease or condition, such as cancer, HIV, malaria, or an autoimmune disease. In still other aspects, administration of a therapeutically effective amount of the fusion proteins described herein is sufficient to act as an adjuvant to increase effectiveness of a vaccine. In yet other aspects, administration of a therapeutically effective amount of the fusion proteins described herein is sufficient to prevent acquisition of a disease or an infection.

Moreover, a treatment regime of a subject with a therapeutically effective amount may consist of a single administration, or alternatively comprise a series of applications. The length of the treatment period depends on a variety of factors, such as the immunogen, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The fusion proteins described herein may, in aspects, be administered before, during or after treatment with conventional therapies for the disease or disorder in question, such as malaria, HIV or cancer. For example, the fusion proteins described herein may find particular use in combination with immunotherapies for treating cancer.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

The term “adjuvant” refers to a compound or mixture that is present in a vaccine and enhances the immune response to an antigen present in the vaccine. For example, an adjuvant may enhance the immune response to a polypeptide present in a vaccine as contemplated herein, or to an immunogenic fragment or variant thereof as contemplated herein. An adjuvant can serve as a tissue depot that slowly releases the antigen and also as a lymphoid system activator that non-specifically enhances the immune response. Examples of adjuvants which may be employed include MPL-TDM adjuvant (monophosphoryl Lipid A/synthetic trehalose dicorynomycolate, e.g., available from GSK Biologics). Another suitable adjuvant is the immunostimulatory adjuvant AS021/AS02 (GSK). These immunostimulatory adjuvants are formulated to give a strong T cell response and include QS-21, a saponin from Quillay saponaria, the TL4 ligand, a monophosphoryl lipid A, together in a lipid or liposomal carrier. Other adjuvants include, but are not limited to, nonionic block co-polymer adjuvants (e.g., CRL 1005), aluminum phosphates (e.g., AIPO.sub.4), R-848 (a Th1-like adjuvant), imiquimod, PAM3CYS, poly (I:C), loxoribine, BCG (bacille Calmette-Guerin) and Corynebacterium parvum, CpG oligodeoxynucleotides (ODN), cholera toxin derived antigens (e.g., CTA 1-DD), lipopolysaccharide adjuvants, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions in water (e.g., MF59 available from Novartis Vaccines or Montanide ISA 720), keyhole limpet hemocyanins, and dinitrophenol.

“Variants” are biologically active fusion proteins, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

“Active” or “activity” for the purposes herein refers to a biological and/or an immunological activity of the fusion proteins described herein, wherein “biological” activity refers to a biological function (either inhibitory or stimulatory) caused by the fusion proteins.

The fusion proteins described herein may include modifications. Such modifications include, but are not limited to, conjugation to an effector molecule such as an anti-malaria agent or an adjuvant. Modifications further include, but are not limited to conjugation to detectable reporter moieties. Modifications that extend half-life (e.g., pegylation) are also included. Proteins and non-protein agents may be conjugated to the fusion proteins by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR) 25, 508-514 (1991), both of which are incorporated by reference herein.

### Fusion Proteins

Described herein are fusion proteins. The fusion proteins comprise a first nanocage monomer subunit of a nanocage monomer linked to a bioactive moiety. The fusion protein, together with a protein comprising a second nanocage monomer subunit, self-assembles to form a nanocage monomer. A plurality of such pairs of fusion proteins self-assemble to form a nanocage. In this way, the bioactive moiety may decorate the interior surface of the assembled nanocage, the exterior surface of the assembled nanocage, or both.

The bioactive moiety may be any moiety capable of being a part of a fusion protein and is, typically a protein. Typically, the bioactive moiety comprises an antibody or fragment thereof, an antigen, a detectable moiety, a pharmaceutical agent, a diagnostic agent, or combinations thereof.

When the bioactive moiety is an antibody and fragment thereof, it may comprise, for example, one or both chains of an Fc fragment. The Fc fragment may be derived from any type of antibody as will be understood but is, typically, an gG1 Fc fragment. The Fc fragment may further comprises one or more mutations, such as LS, YTE, LALA, and/or LALAP, that modulate the half-life of the fusion protein and/or the resulting assembled nanocage comprising the fusion protein. For example, the half-life may be in the scale of minutes, days, weeks, or even months.

Moreover, other substitutions in the fusion proteins and nanocages described herein are contemplated, including Fc sequence modifications and addition of other agents (e.g. human serum albumin peptide sequences), that allow changes in bioavailability and will be understood by a skilled person. Furthermore, the fusion proteins and nanocages described herein can be modulated in sequence or by addition of other agents to mute immunogenicity and anti-drug responses (therapeutic, e.g. matching sequence to host, or addition of immunosuppressive therapies [such as, for example, methotrexate when administering infliximab for treating rheumatoid arthritis or induction of neonatal tolerance, which is a primary strategy in reducing the incidence of inhibitors against FVIII (reviewed in: DiMichele DM, Hoots W K, Pipe S W, Rivard G E, Santagostino E. International workshop on immune tolerance induction: consensus recommendations. Haemophilia. 2007; 13:1-22, incorporated herein by reference in its entirety]), or to enhance immune responses (e.g. bacterial sequences for vaccines).

In other aspects, when the bioactive moiety is an antibody or fragment thereof, it may comprise, for example, a heavy and/or light chain of a Fab fragment. The antibody or fragment thereof may comprise a scFab fragment, a scFv fragment, or a sdAb fragment, for example. It will be understood that any antibody or fragment thereof may be used in the fusion proteins described herein.

Generally, the fusion protein described herein is associated with a Fab light chain and/or heavy chain, which may be produced separately or contiguously with the fusion protein.

In cases where the antibody or fragment thereof comprises two chains, such as a first and second chain in the case of a Fc fragment, or a heavy and light chain, the two chains are optionally separated by a linker. The linker may be flexible or rigid, but it typically flexible to allow the chains to fold appropriately. The linker is generally long enough to impart some flexibility to the fusion protein, although it will be understood that linker length will vary depending upon the nanocage monomer and bioactive moiety sequences and the three-dimensional conformation of the fusion protein. Thus, the linker is typically from about 1 to about 30 amino acid residues, such as from about 1, 2, 3, 4, 5, 6, 7, 8, 9, 10, 11, 12, 13, 14, 15, 16, 17, 18, 19, 20, 21, 22, 23, 24, 25, 26, 27, 28, or 29 to about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues, such as from about 8 to about 16 amino acid residues, such as 8, 10, or 12 amino acid residues.

The linker may be of any amino acid sequence and, in one typical example, the linker comprises a GGS repeat and, more typically, the linker comprises about 2, 3, 4, 5, or 6 GGS repeats, such as about 4 GGS repeats. In specific aspects, the linker comprises or consists of a sequence at least 70% (such as at least 75%, 80%, 85%, 90%, 95%, 96%, 97%, 98%, 99%, or 100%) identical to:

GGSSGSGSGSTGTSSSGTGTSAGTTGTSASTSGSGSGGGGGSGGGGSAG
GTATAGASSGSGSSGSSSSGGTG.

In typical aspects, the antibody or fragment thereof binds specifically to an antigen associated with an antibody-preventable and/or antibody-treatable condition. For example, the antigen to which the antibody or fragment thereof binds may be associated with an infectious agent, including a virus (e.g., HIV, including HIV-1, influenza, RSV, rotavirus), bacteria (e.g., TB, C. difficile) parasite (e.g., malaria), fungus, or yeast, a cancer (e.g., CD19, CD22, CD79, BCMA, or CD20), including solid and liquid cancers, or an immune disease, including an autoimmune disease. Typically, the antigen is associated with HIV-1 and the antibody or fragment thereof comprises, for example, lbalizumab-A12P, 10E8, 10E8.v4, N49P7, PGDM1400, 10-1074, VRC01, or combinations thereof.

In a specific example, the antibody or fragment thereof comprises or consists of a sequence at least 70% (such as at least 75%, 80%, 85%, 90%, 95%, 96%, 97%, 98%, 99%, or 100%) identical to one or more of the following sequences:

Fc chain 1:
DKTHTCPPCPAPELLGGPSVFLFPPKPKDTLMISRTPEVTCVVDVSHED
PEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRW
QQGNVFSCSVLHEALHSHYTQKSLSLSPGK;
Fc chain 2:
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVLHEALHSHYTQKSLSLSPGK;
Ibalizumab-A12P light chain:
DIVMTQSPDSLPVSLGERVTMNCKSSQSLLYSTNQKNYLAWYQQKPGQS
PKLLIYWASTRESGVPDRFSGSGSGTDFTLTISSVQAEDVAVYYCQQYY
SYRTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC;
Ibalizumab-A12P heavy chain:
QVQLQQSGPEVVKPGASVKMSCKASGYTFTSYVIHWVRQKPGQGLDWIG
YINPYNDGTDYDEKFKGKATLTSDTSTSTAYMELSSLRSEDTAVYYCAR
EKDNYATGAWFAYWGQGTLVTVSSASTKGPSVFPLAPCSRSTSESTAAL
GCLVKDYFPEPVTVSWNSGALTSGVHTFPAVLQSSGLYSLSSVVTVPSS
SLGTKTYTCNVDHKPSNTKVDKRVESKYGSR;
10E8.v4 light chain:
SELTQDPAVSVALKQTVTITCRGDSLRSHYASWYQKKPGQAPVLLFYGK
NNRPSGIPDRFSGSASGNRASLTITGAQAEDEADYYCSSRDKSGSRLSV
FGGGTKLTVLSQPKAAPSVTLFPPSSEELQANKATLVCLISDFYPGAVT
VAWKADSSPVKAGVETTTPSKQSNNKYAASSYLSLTPEQWKSHRSYSCQ
VTHEGSTVEKTVAPTEC;
10E8.v4 heavy chain:
EVRLVESGGGLVKPGGSLRLSCSASGFDFDNAWMTWVRQPPGKGLEWVG
RITGPGEGWSVDYAESVKGRFTISRDNTKNTLYLEMNNVRTEDTGYYFC
ARTGKYYDFWSGYPPGEEYFQDWGQGTLVIVSSASTKGPSVFPLAPSSK
STSGGTAALGCLVKDYFPEPVTVSWNSGALTSGVHTFPAVLQSSGLYSL
SSVVTVPSSSLGTQTYICNVNHKPSNTKVDKKVEPKSCSR;
N49P7 light chain:
QSALTQPRSVSASPGQSVTISCTGTHNLVSWCQHQPGRAPKLLIYDFNK
RPSGVPDRFSGSGSGGTASLTITGLQDDDDAEYFCWAYEAFGGGTKLTV
LGQPKAAPSVTLFPPSSEELQANKATLVCLISDFYPGAVTVAWKADSSP
VKAGVETTTPSKQSNNKYAASSYLSLTPEQWKSHRSYSCQVTHEGSTVE
KTVAPTEC;
N49P7 heavy chain:
ADLVQSGAVVKKPGDSVRISCEAQGYRFPDYIIHWIRRAPGQGPEWMGW
MNPMGGQVNIPWKFQGRVSMTRDTSIETAFLDLRGLKSDDTAVYYDRSN
GSGKRFESSNWFLDLWGRGTAVTIQSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDSR;
PGDM1400 light chain:
DFVLTQSPHSLSVTPGESASISCKSSHSLIHGDRNNYLAWYVQKPGRSP
QLLIYLASSRASGVPDRFSGSGSDKDFTLKISRVETEDVGTYYCMQGRE
SPWTFGQGTKVDIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC;
PGDM1400 heavy chain:
QAQLVQSGPEVRKPGTSVKVSCKAPGNTLKTYDLHWVRSVPGQGLQWMG
WISHEGDKKVIVERFKAKVTIDWDRSTNTAYLQLSGLTSGDTAVYYCAK
GSKHRLRDYALYDDDGALNWAVDVDYLSNLEFWGQGTAVTVSSASTKGP
SVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCD
SR;

or combinations thereof.

In further aspects, the antibody or fragment thereof is conjugated to or associated with a further moiety, such as an antigen, a detectable moiety (e.g., a small molecule, fluorescent molecule, radioisotope, or magnetic particle), a pharmaceutical agent, a diagnostic agent, or combinations thereof and may comprise, for example, an antibody-drug conjugate.

In aspects wherein the bioactive moiety is an antigen, the antigen may be associated with a vaccine-preventable and/or vaccine-treatable condition, for example. In such cases, the antigen may be associate with, for example, an infectious agent, including a virus, bacteria, parasite, fungus, or yeast, a cancer, including solid and liquid cancers, or an immune disease, including an autoimmune disease.

In aspects wherein the bioactive moiety is a detectable moiety, the detectable moiety may comprise a fluorescent protein, such as GFP, EGFP, Ametrine, and/or a flavin-based fluorescent protein, such as a LOV-protein, such as iLOV.

In aspects wherein the bioactive moiety is a pharmaceutical agent, the pharmaceutical agent may comprise for example, a small molecule, peptide, lipid, carbohydrate, or toxin.

In typical aspects, the nanocage assembled from the fusion proteins described herein comprises from about 3 to about 100 nanocage monomers, such as from about 3, 4, 5, 6, 7, 8, 9, 10, 12, 14, 16, 18, 20, 22, 24, 26, 28, 30, 32, 34, 36, 38, 40, 42, 44, 46, 48, 50, 52, 55, 56, 58, 60, 62, 64, 66, 68, 70, 72, 74, 76, 78, 80, 82, 84, 86, 88, 90, 92, 94, 96, or 98 to about 4, 5, 6, 7, 8, 9, 10, 12, 14, 16, 18, 20, 22, 24, 26, 28, 30, 32, 34, 36, 38, 40, 42, 44, 46, 48, 50, 52, 55, 56, 58, 60, 62, 64, 66, 68, 70, 72, 74, 76, 78, 80, 82, 84, 86, 88, 90, 92, 94, 96, 98, or 100 nanocage monomers, such as 24, 32, or 60 monomers. The nanocage monomer may be any known nanocage monomer, natural, synthetic, or partly synthetic and is, in aspects, selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof. Typically, the nanocage monomer is ferritin or apoferritin.

When apoferritin is chosen as the nanocage monomer, typically the first and second nanocage monomer subunits interchangeably comprise the “N” and “C” regions of apoferritin. It will be understood that other nanocage monomers can be divided into bipartite subunits much like apoferritin as described herein so that the subunits self-assemble and are each amenable to fusion with a bioactive moiety.

Typically, the “N” region of apoferritin comprises or consists of a sequence at least 70% (such as at least 75%, 80%, 85%, 90%, 95%, 96%, 97%, 98%, 99%, or 100%) identical to:

MSSQIRQNYSTDVEAAVNSLVNLYLQASYTYLSLGFYFDRDDVALEGVS
HFFRELAEEKREGYERLLKMQNQRGGRALFQDIKKPAEDEW.

Typically, the “C” region of apoferritin comprises or consists of a sequence at least 70% (such as at least 75%, 80%, 85%, 90%, 95%, 96%, 97%, 98%, 99%, or 100%) identical to:

GKTPDAMKAAMALEKKLNQALLDLHALGSARTDPHLCDFLETHFLDEEV
KLIKKMGDHLTNLHRLGGPEAGLGEYLFERLTLRHD.

In aspects, the fusion protein described herein, further comprises a linker between the nanocage monomer subunit and the bioactive moiety, much like the linker described above. Again, the linker may be flexible or rigid, but it typically flexible to allow the bioactive moiety to retain activity and to allow the pairs of nanocage monomer subunits to retain self-assembly properties. The linker is generally long enough to impart some flexibility to the fusion protein, although it will be understood that linker length will vary depending upon the nanocage monomer and bioactive moiety sequences and the three-dimensional conformation of the fusion protein. Thus, the linker is typically from about 1 to about 30 amino acid residues, such as from about 1, 2, 3, 4, 5, 6, 7, 8, 9, 10, 11, 12, 13, 14, 15, 16, 17, 18, 19, 20, 21, 22, 23, 24, 25, 26, 27, 28, or 29 to about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues, such as from about 8 to about 16 amino acid residues, such as 8, 10, or 12 amino acid residues.

The linker may be of any amino acid sequence and, in one typical example, the linker comprises a GGS repeat and, more typically, the linker comprises about 2, 3, 4, 5, or 6 GGS repeats, such as about 4 GGS repeats. In specific aspects, the linker comprises or consists of a sequence at least 70% (such as at least 75%, 80%, 85%, 90%, 95%, 96%, 97%, 98%, 99%, or 100%) identical to:

ASTASSASSGGGGGGSGGSGGSGGS

Similarly, the fusion protein may further comprising a C-terminal linker for improving one or more attributes of the fusion protein. In aspects, the comprises a GGS repeat and, more typically, the linker comprises about 2, 3, 4, 5, or 6 GGS repeats, such as about 4 GGS repeats. In specific aspects, the C-terminal linker comprises or consists of a sequence at least 70% (such as at least 75%, 80%, 85%, 90%, 95%, 96%, 97%, 98%, 99%, or 100%) identical to:

GGSGGSGGSGGSGGGASGGS.

Also described herein is a pair of the fusion proteins described above, wherein the pair self-assembles to form a nanocage monomer, wherein the first and second nanocage monomer subunits are fused to different bioactive moieties. This provides multivalency and/or multispecificity to a single nanocage monomer assembled from the pair of subunits.

In certain aspects, the fusion protein may further comprise an antigen. Such aspects are described explicitly in International Patent Application No. WO 2019/023812, which is incorporated herein by reference in its entirety. Briefly, in such aspects, the antigen has at least a first and a second antibody-binding epitope; and an antibody or fragment thereof that is specific for at least the first antigen epitope. Binding of the antibody or fragment thereof to the first antigen epitope presents the second antigen epitope for binding to an antigen-binding moiety and/or the first antibody-binding epitope binds to the antibody or fragment thereof and wherein said binding presents said second antibody-binding epitope in the context of the antibody or fragment thereof.

In other aspects, the antibody or fragment thereof may be directed to any antigen, such as those listed above. Typically, the antigen is derived from a cancer or an infectious agent such as hepatitis A, B, C, HIV, mycobacteria, malaria pathogens, SARS pathogens, herpesvirus, influenzavirus, poliovirus or from bacterial pathogens such as chlamydia and mycobacteria, or from autoreactive B cells or any T cells for co-recruitment and cytotoxic killing.

The fusion proteins described herein may alternatively find use as therapeutics or diagnostic agents. Thus, the antibody or fragment thereof in aspects may be specific for a tumour antigen or an autoantigen, for example.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The polypeptides or fusion proteins of the present invention may also comprise additional sequences to aid in their expression, detection or purification. Any such sequences or tags known to those of skill in the art may be used. For example, and without wishing to be limiting, the fusion proteins may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK, Xpress tag, Avi tag,Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a His₅ or His₆), or a combination thereof.

In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

More specifically, a tag cassette may comprise an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv or scFab) to an effector domain, (e) interposed between and connecting subunits of a binding domain, or (f) at the amino-terminus of a single chain fusion protein. In certain embodiments, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

Also encompassed herein are isolated or purified fusion proteins, polypeptides, or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, a film, or any other useful surface.

In other aspects, the fusion proteins may be linked to a cargo molecule; the fusion proteins may deliver the cargo molecule to a desired site and may be linked to the cargo molecule using any method known in the art (recombinant technology, chemical conjugation, chelation, etc.). The cargo molecule may be any type of molecule, such as a therapeutic or diagnostic agent. For example, and without wishing to be limiting in any manner, the therapeutic agent may be a radioisotope, which may be used for radioimmunotherapy; a toxin, such as an immunotoxin; a cytokine, such as an immunocytokine; a cytotoxin; an apoptosis inducer; an enzyme; an anti-cancer antibody for immunotherapy; or any other suitable therapeutic molecule known in the art. In the alternative, a diagnostic agent may include, but is by no means limited to a radioisotope, a paramagnetic label such as gadolinium or iron oxide, a fluorophore, a Near Infra-Red (NIR) fluorochrome or dye (such as Cy3, Cy5.5, Alexa680, Dylight680, or Dylight800), an affinity label (for example biotin, avidin, etc), fused to a detectable protein-based molecule, or any other suitable agent that may be detected by imaging methods. In a specific, non-limiting example, the fusion protein may be linked to a fluorescent agent such as FITC or may genetically be fused to the Enhanced Green Fluorescent Protein (EGFP).

In some aspects, the cargo molecule is a protein and is fused to the fusion protein such that the cargo molecule is contained in the nanocage internally. In other aspects, the cargo molecule is not fused to the fusion protein and is contained in the nanocage internally. The cargo molecule is typically a protein, a small molecule, a radioisotope, or a magnetic particle.

The fusion proteins described herein specifically bind to their targets. Antibody specificity, which refers to selective recognition of an antibody for a particular epitope of an antigen, of the antibodies or fragments described herein can be determined based on affinity and/or avidity. Affinity, represented by the equilibrium constant for the dissociation of an antigen with an antibody (Ko), measures the binding strength between an antigenic determinant (epitope) and an antibody binding site. Avidity is the measure of the strength of binding between an antibody with its antigen. Antibodies typically bind with a Ko of 10⁻⁵ to 10⁻¹¹ M. Any Ko greater than 10⁻⁴ M is generally considered to indicate non-specific binding. The lesser the value of the K_{D}, the stronger the binding strength between an antigenic determinant and the antibody binding site. In aspects, the antibodies described herein have a K_{D} of less than 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M.

Also described herein are nanocages comprising at least one fusion protein described herein and at least one second nanocage monomer subunit that self-assembles with the fusion protein to form a nanocage monomer. Further, pairs of the fusion proteins are described herein, wherein the pair self-assembles to form a nanocage monomer and wherein the first and second nanocage monomer subunits are fused to different bioactive moieties.

It will be understood that the nanocages may self-assemble from multiple identical fusion proteins, from multiple different fusion proteins (and therefore be multivalent and/or multispecific), from a combination of fusion proteins and wild-type proteins, and any combination thereof. For example, the nanocages may be decorated internally and/or externally with at least one of the fusion proteins described herein in combination with at least one anti-cancer antibody for immunotherapy. In typical aspects, from about 20% to about 80% of the nanocage monomers comprise the fusion protein described herein. In view of the modular solution described herein, the nanocages could in theory comprise up to twice as many bioactive moieties as there are monomers in the nanocage, as each nanocage monomer may be divided into two subunits, each of which can independently bind to a different bioactive moiety. It will be understood that this modularity can be harnessed to achieve any desired ratio of bioactive moieties as described herein in specific example to a 4:2:1:1 ratio of four different bioactive moieties. For example, the nanocages described herein may comprise at least 2, 3, 4, 5, 6, 7, 8, 9, or 10 different bioactive moieties. In this way, the nanocages can be multivalent and/or multispecific and the extent of this can be controlled with relative ease.

In aspects, the nanocages described herein may further comprise at least one whole nanocage monomer, optionally fused to a bioactive moiety that may be the same or different from the bioactive moiety described herein as being linked to a nanocage monomer subunit.

In typical aspects, the nanocages described herein comprise a first, second, and third fusion protein, and at least one whole nanocage monomer, optionally fused to a bioactive moiety, wherein the bioactive moieties of the first, second, and third fusion proteins and of the whole nanocage monomer are all different from one another.

More typically, the first, second, and third fusion proteins each comprise an antibody or fragment thereof fused to N- or C-ferritin, wherein at least one of the first, second, and third fusion proteins is fused to N-ferritin and at least one of the first, second, and third fusion proteins is fused to C-ferritin. For example, the antibody or fragment thereof of the first fusion protein is typically an Fc fragment; the second and third fusion proteins typically each comprise an antibody or fragment thereof specific for a different antigen of a virus such as HIV or one of the second and third fusion proteins comprises an antibody or fragment thereof specific for an antigen of a virus such as HIV and the third fusion protein comprises an antibody or fragment thereof specific for a different antigen, such as the CD4 receptor; and the whole nanocage monomer is fused to a bioactive moiety that is specific for another different antigen, optionally of the same virus such as HIV.

In aspects, the antibody or fragment thereof of the second fusion protein is N49P7 or iMab A12P; and the antibody or fragment thereof of the third fusion protein is 10E8v4. In a typical aspect, the nanocage described herein comprises the following four fusion proteins, optionally in a 4:2:1:1: ratio:

a. PGDM1400 (optionally scPGDM1400) fused to full length ferritin;

b. Fc (optionally scFc) fused to N-ferritin;

c. N49P7 or iMab A12P (optionally scN49P7 or sciMab A12P) fused to C-ferritin; and

d. 10E8v4 (optionally sc10E8v4) fused to C-ferritin.

In aspects, the nanocage described herein comprises or consists of sequences at least 70% (such as at least 75%, 80%, 85%, 90%, 95%, 96%, 97%, 98%, 99%, or 100%) identical to one or more of the following sequences, where ferritin subunits are in bold, linkers are underlined, light chains are italicized, and heavy chains are in lowercase:

a. PGDM1400-hFerr:
DFVLTQSPHSLSVTPGESASISCKSSHSLIHGDRNNYLAWYVQKPGRSP
QLLIYLASSRASGVPDRFSGSGSDKDFTLKISRVETEDVGTYYCMQGRE
SPWTFGQGTKVDIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGECGGSSGSGSGSTGTSSSGTGTSAGTTG
TSASTSGSGSGGGGGSGGGGSAGGTATAGASSGSGSSGSSSSGGTGqaq
lvqsgpevrkpgtsvkvsckapgntlktydlhwvrsvpgqglqwmgwis
hegdkkviverfkakvtidwdrstntaylqlsgltsgdtavyycakgsk
hrlrdyalydddgalnwavdvdylsnlefwgqgtavttvssastkgpsv
fplapsskstsggtaalgclvkdyfpepvtvswnsgaltsgvhtfpavl
qssglyslssvvtvpssslgtqtyivnvnhkpsntkvdkkvepkscdsr
ASTASSASSGGGGGSGGSGGSGGSMSSQIRQNYSTDVEAAVNSLVNLYL
QASYTYLSLGFYFDRDDVALEGVSHFFRELAEEKREGYERLLKMQNQRG
GRALFQDIKKPAEDEWGKTPDAMKAAMALEKKLNQALLDLHALGSARTD
PHLCDFLETHFLDEEVKLIKKMGDHLTNLHRLGGPEAGLGEYLFERLTL
RHDGGSGGSGGSGGSGGGASGGS;
b. Fc-N-hFerr LS
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVLHEALHSHYTQKSLSLSPGKGGSSGSGSGSTGTSSSGT
GTSAGTTGTSASTSGSGSGGGGGSGGGGSAGGTATAGASSGSGSSGSSS
SGGTGdkthtcppcpapellggpsvflfppkpkdtlmisrtpevtcvvv
dvshedpevkfnwyvdgvevhnaktkpreeqynstyrvvsvltvlhqdw
lngkeykckvsnkalpapiektiskakgqprepqvytlppsreemtknq
vsltclvkgfypsdiavewesngqpennykttppvdlsdgsfflysklt
vdksrwqqgnvfscsvlhealhshytqkslslspgksrASTASSASSGG
GGGGSGGSGGSGGSMSSQIRQNYSTDVEAAVNSLVNLYLQASYTYLSLG
FYFDRDDVALESGVSHFFRELAEEKREGYERLLKMQNQRGGRALFQDIK
KPAEDEW;
c1. N49P7-C-hFerr
QSALTQPRSVSASPGQSVTISCTGTHNLVSWCQHQPGRAPKLLIYDFNK
RPSGVPDRFSGSGSGGTASLTITGLQDDDDAEYFCWAYEAFGGGTKLTV
LGQPKAAPSVTLFPPSSEELQANKATLVCLISDFYPGAVTVAWKADSSP
VKAGVETTTPSKQSNNKYAASSYLSLTPEQWKSHRSYSCQVTHEGSTVE
KTVAPTECGGSSGSGSGSTGTSSSGTGTSAGTTGTSASTSGSGSGGGGG
SGGGGSAGGTATAGASSGSGSSGSSSSGGTGadlvqsgavvkkpgdsvr
isceaqgyrfpdyiihwirrapgqgpewmgwmnpmggqvnipwkfqgrv
smtrdtsietafldlrglksddtavyycvrdrsngsgkrfessnwfldl
wgrgtavtiqsastkgpsvfplapsskstsggtaalgclvkdyfpepvt
vswnsgaltsgvhtfpavlqssglyslssvvtvpssslgtqtyicnvnh
kpsntkvdkkvepkscdsrASTASSASSGGGGGGSGGSGGSGGSGKTPD
AMKAAMALEKKLNQALLDLHALGSARTDPHLCDFLETHFLDEEVKLIKK
MGDHLTNLHRLGGPEAGLGEYLFERLTLRHDGGSGGSGGSGGSGGGASG
GS;
c2. Ibalizumab-A12P-C-hFerr
DIVMTQSPDSLPVSLGERVTMNCKSSQSLLYSTNQKNYLAWYQQKPGQS
PKLLIYWASTRESGVPDRFSGSGSGTDFTLTISSVQAEDVAVYYCQQYY
SYRTFGGGTKLEIKRTVAAPSVFIFPPSDEQLKSGTASSVVCLLNNFYP
REAKVQWKVDNALQSGNSQESVTEQDSKDSTYSLSSTLTLSKADYEKHK
VYACEVTHQGLSSPVTKSFNRGECGGSSGSGSGSTGTSSSGTGTSAGTT
GTSASTSGSGSGGGGGSGGGGSAGGTATAGASSGSGSSGSSSSGGTGqv
qlqqsgpevvkpgasvkmsckasgytftsyvihwvrqkpgqgldwigyi
npyndgtdydekfkgkatltsdtststaymelsslrsedtavyycarek
dnyatgawfaywgqgtlvtvssastkgpsvfplapcsrstsestaalgc
lvkdyfpepvtvswnsgaltsgvhtfpavlqssglyslssvvtvpsssl
gtktytcnvdhkpsntkvdkrveskygsrASTASSASSGGGGGGSGGSG
GSGGSGKTPDAMKAAMALEKKLNQALLDLHALGSARTDPHLCDFLETHF
LDEEVKLIKKMGDHLTNLHRLGGPEAGLGEYLFERLTLRHDGGSGGSGG
SGGSGGGASGGS;
or
d. 10E8.v4-C-hFerr
SELTQDPAVSVALKQTVTITCRGDSLRSHYASWYQKKPGQAPVLLFYGK
NNRPSGIPDRFSGSASGNRASLTITGAQAEDEADYYCSSRDKSGSRLSV
FGGGTKLTVLSQPKAAPSVTLFPPSSEELQANKATLVCLISDFYPGAVT
VAWKADSSPVKAGVETTTPSKQSNNKYAASSYLSLTPEQWKSHRSYSCQ
VTHEGSTVEKTVAPTECGGSSGSGSGSTGTSSSGTGTSAGTTGTSASTS
GSGSGGGGGSGGGGSAGGTATAGASSGSGSSGSSSSGGTGevrlvesgg
glvkpggslrlscsasgfdfdnawmtwvrqppgklgewvgritgpgegw
svdyaesvkgrftisrdntkntlylemnnvrtedtgyyfcartgkyydf
wsgyppgeeyfqdwgqgtlvivssastkgpsvfplalsskstsggtaal
gclvkdyfpepvtvswnsgaltsgvhtfpavlqssglyslssvvtvpss
slgtqtyicnvnhkpsntkvdkkvepkscsrASTASSASSGGGGGGSGG
SGGSGGSGKTPDAMKAAMALEKKLNQALLDLHALGSARTDPHLCDFLET
HFLDEEVKLIKKMGDHLTNLHRLGGPEAGLGEYLFERLTLRHDGGSGGS
GGSGGSGGGASGGS.

It will be understood that generally the nanocages described herein are hollow and therefore capable of carrying a cargo molecule, such as a pharmaceutical agent, a diagnostic agent, and/or an imaging agent. Generally, the cargo molecule is not fused to the fusion protein and is contained in the nanocage internally, however, the cargo molecule may alternatively be a protein and fused to the fusion protein such that the cargo molecule is contained in the nanocage internally.

In aspects, the cargo molecule is contained internally to provide T-cell epitopes, but optionally not B-cell epitopes. Alternatively, the cargo molecule is fused to the fusion protein and contained internally to provide T-cell epitopes, but optionally not B-cell epitopes.

The cargo molecule may be a fluorescent protein, such as GFP, EGFP, Ametrine, and/or a flavin-based fluorescent protein, such as a LOV-protein, such as iLOV and/or the cargo molecule may be a small molecule, radioisotope, or magnetic particle.

Furthermore, the nanocage may further comprise an antigen on the surface, which may be expressed as a fusion protein with a nanocage monomer.

Also described herein are vaccines comprising the nanocage described herein, as well as compositions comprising the nanocage, such as therapeutic or prophylactic compositions. Related methods and uses for treating and/or preventing a disease or condition are also described, wherein the method or use comprises administering the nanocage, vaccine, or composition described herein to a subject in need thereof. The nanocages can be used for treatment of any disease or condition in which bioactive therapy or, more specifically, antibody therapy may find use, but for example, the disease or condition is typically cancer, an infectious disease such as HIV, malaria, influenza, RSV, rotavirus, or an autoimmune disease.

Also described herein are nucleic acid molecules encoding the fusion proteins and polypeptides described herein, as well as vectors comprising the nucleic acid molecules and host cells comprising the vectors.

Polynucleotides encoding the fusion proteins described herein include polynucleotides with nucleic acid sequences that are substantially the same as the nucleic acid sequences of the polynucleotides of the present invention. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

Suitable sources of polynucleotides that encode fragments of antibodies include any cell, such as hybridomas and spleen cells, that express the full-length antibody. The fragments may be used by themselves as antibody equivalents, or may be recombined into equivalents, as described above. The DNA deletions and recombinations described in this section may be carried out by known methods, such as those described in the published patent applications listed above in the section entitled “Functional Equivalents of Antibodies” and/or other standard recombinant DNA techniques, such as those described below. Another source of DNAs are single chain antibodies produced from a phage display library, as is known in the art.

Additionally, expression vectors are provided containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colEI, pCRI, pBR322, pMB9, pUC, pKSM, and RP4. Prokaryotic vectors also include derivatives of phage DNA such as MI3 and other filamentous single-stranded DNA phages. An example of a vector useful in yeast is the 2p plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P J. Southern & P. Berg, J. Mol. Appl. Genet, 1:327-341 (1982); Subramani et al, Mol. Cell. Biol, 1: 854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors typically contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Also described herein are recombinant host cells containing the expression vectors previously described. The fusion proteins described herein can be expressed in cell lines other than in hybridomas. Nucleic acids, which comprise a sequence encoding a polypeptide according to the invention, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces.

These present recombinant host cells can be used to produce fusion proteins by culturing the cells under conditions permitting expression of the polypeptide and purifying the polypeptide from the host cell or medium surrounding the host cell. Targeting of the expressed polypeptide for secretion in the recombinant host cells can be facilitated by inserting a signal or secretory leader peptide-encoding sequence (See, Shokri et al, (2003) Appl Microbiol Biotechnol. 60(6): 654-664, Nielsen et al, Prot. Eng., 10:1-6 (1997); von Heinje et al., Nucl. Acids Res., 14:4683-4690 (1986), all of which are incorporated by reference herein) at the 5′ end of the antibody-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium.

The fusion proteins described herein can be fused to additional amino acid residues. Such amino acid residues can be a peptide tag to facilitate isolation, for example. Other amino acid residues for homing of the antibodies to specific organs or tissues are also contemplated.

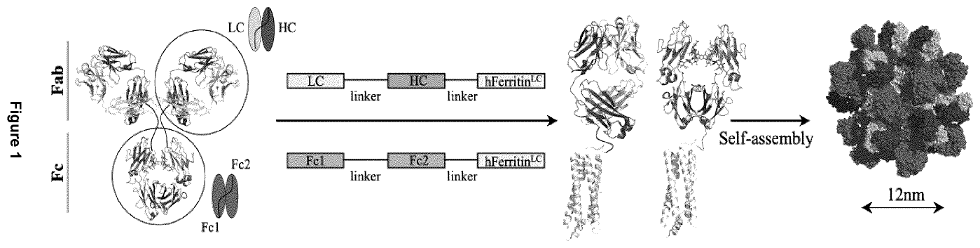

It will be understood that a Fab-nanocage can be generated by co-transfection of HC-ferritin and LC. Alternatively, single-chain Fab-ferritin nanocages can be used that only require transfection of one plasmid, as shown in FIG. 1C. This can be done with linkers of different lengths between the LC and HC for example 60 or 70 amino acids. When single-chain Fabs are used, it can be ensured that the heavy chain and light chain are paired. Tags (e.g. Flag, HA, myc, His6x, Strep, etc.) can also be added at the N terminus of the construct or within the linker for ease of purification as described above. Further, a tag system can be used to make sure many different Fabs are present on the same nanoparticle using serial/additive affinity chromatography steps when different Fab-nanoparticle plasmids are co-transfected. This provides multi-specificity to the nanoparticles. Protease sites (e.g. TEV, 3C, etc.) can be inserted to cleave linkers and tags after expression and/or purification, if desired. An example of such a construct is for anti-HIV broadly neutralizing scFab 10E8:

YELTQETGVSVALGRTVTITCRGDSLRSHYASWYQKKPGQAPILLFYGK
NNRPSGVPDRFSGSASGNRASLTISGAQAEDDAEYYCSSRDKSGSRLSV
FGGGTKLTVLSQPKAAPSVTLFPPSSEELQANKATLVCLISDFYPGAVT
VAWKADSSPVKAGVETTTPSKQSNNKYAASSYLSLTPEQWKSHRSYSCQ
VTHEGSTVEKTVAPTECGGSSGSGSGSTGENLYFQGSAGTTGTSASTSG
YPYDVPDYAGGGGSAGGTATLEVLFQGPSSGSSSSGGTGEVQLVESGGG
LVKPGGSLRLSCSASGFDFDNAWMTWVRQPPGKGLEWVGRITGPGEGWS
VDYAAPVEGRFTISRLNSINFLYLEMNNLRMEDSGLYFCARTGKYYDFW
SGYPPGEEYFQDWGRGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKRVEPKSCSRGGGGGSGGSGGSGGSMSSQ
IRQNYSTDVEAAVNSLVNLYLQASYTYLSLGFYFDRDDVALEGVSHFFR
ELAEEKREGYERLLKMQNQRGGRALFQDIKKPAEDEWGKTPDAMKAAMA
LEKKLNQALLDLHALGSARTDPHLCDFLETHFLDEEVKLIKKMGDHLTN
LHRLGGPEAGLGEYLFERLTLRHD

In another aspect, described herein are methods of vaccinating subjects by administering a therapeutically effective amount of the fusion proteins described herein to a mammal in need thereof, typically a young, juvenile, or neonatal mammal. Therapeutically effective means an amount effective to produce the desired therapeutic effect, such as providing a protective immune response against the antigen in question.

Any suitable method or route can be used to administer the fusion proteins and vaccines described herein. Routes of administration include, for example, oral, intravenous, intraperitoneal, subcutaneous, or intramuscular administration.

It is understood that the fusion proteins described herein, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

Although human antibodies are particularly useful for administration to humans, they may be administered to other mammals as well. The term “mammal” as used herein is intended to include, but is not limited to, humans, laboratory animals, domestic pets and farm animals.

Also included herein are kits for vaccination, comprising a therapeutically or prophylactically effective amount of a fusion protein described herein. The kits can further contain any suitable adjuvant for example. Kits may include instructions.

The above disclosure generally describes the present invention. A more complete understanding can be obtained by reference to the following specific examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention, and are not to be construed as limiting in any way the remainder of the disclosure.

### EXAMPLES

### Example 1

### Introduction

Despite over 30 years of effort, no effective vaccine or cure exists against the human immunodeficiency virus type I (HIV-1). Encouraging in this quest however is the fact that a small proportion of HIV-1 infected individuals develop antibodies with exceptional neutralization potency across circulating HIV-1 isolates. Since the first generation of broadly neutralizing antibodies (bNAbs) 2F5¹, 4E10^{2,3}, 2G12⁴ and b12^{5,6} were discovered, the catalogue of bNAbs has dramatically increased due to implementation of new technologies of Env-specific single B cell sorting⁷⁻⁹, antibody cloning and high-throughput neutralization assays¹⁰⁻¹³, and more recently proteomic deconvolution¹⁴. Several dozens of HIV bNAbs have now been described to target six conserved sites on the trimeric HIV Envelope (Env), including the V1/V2 loops at the trimer apex, V3 loop glycans, the CD4 binding site (CD4bs), the gp120-g41 interface, the fusion peptide and the membrane-proximal external region (MPER)^{7,9,19,10,12-18}.

The interest of bNAbs as therapeutic molecules in the fight against HIV-1 arise from the potent antiviral activity observed for some in challenge studies in macaques²⁰⁻²⁴ and humanized mice²⁵⁻²⁵, and from the reduced viremia achieved in infected humans when bNAbs are therapeutically infused²⁹⁻³³. In addition, antibodies possess key advantages in comparison to oral antiretroviral therapy (ART): they have longer circulating half-lives and can form immune complexes that enhance host immunity to the virus. These observations have led to the clinical evaluation of antibody-based therapy to confer protection against HIV-1 through passive administration of bNAbs instead of or in addition to conventional vaccinology, and to efforts to control and/or clear HIV-1 in infected individuals.

One of the main limitations for the clinical use of bNAbs is the rapid selection of neutralization-resistant virus populations^{30-32,34,35}. RNA viruses such as HIV exhibit an extraordinary genetic diversity³⁶ enabling the virus to develop resistant mutations to escape mAb recognition. However, mutations that abrogate binding to certain bNAbs can carry a significant penalty in viral fitness³⁷⁻³⁹. Analogous to the combination of different drugs in HIV-1 treatment regimens, this observation suggests that a successful antibody-based therapy against HIV-1 should include a combination of bNAb specificities. As a consequence, the development of different formats of antibody-like molecules with bi-⁴⁰⁻⁴² or tri-specificity⁴³⁻⁴⁵ toward Env has recently been explored. An additional consideration is the amount of antibody required for in vivo efficacy. Indeed, extensive efforts have been directed towards engineering bNAbs to improve their potency using structure-guide design or bioinformatic approaches, such as VRCO1⁴⁸, 10E8^{47,48} and NIH45-46⁴⁹, but so far with moderate success. In the case of bispecific and trispecific antibodies that target multiple epitopes in Env, potency is generally limited by the potency of their parental mAbs. Consequently, a significant improvement in neutralization breadth but relatively little in antiviral potency has so far been achieved^{40,43,44,45}.

The potency of an antibody is greatly affected by its ability to interact with more than one epitope on the same virus⁵⁰⁻⁵². This effect is commonly known as avidity (enhanced apparent affinity) and is a property used in nature by IgM antibodies to compensate for their usually low affinity. Therefore, addition of the mu-tailpiece of the IgM to the constant region of the IgG has been explored to generate dodeca-valency IgM-like molecules with improved bioactivity^{53,54}. Surpassing evolution, a variety of unnatural antibody formats have been engineered to overcome the limitation of IgG bivalency. Some of these designs include tandem fusions of Fabs in a linear head-to-tail manner⁵⁵, diabody combination in tandem (Tamdabs)⁵⁶ or fused to the CH3 of an IgG (di-diabody)⁵⁷, appended IgGs⁵⁸⁻⁶⁰ and the use of multimerization scaffolds such as p53⁶¹, leucine zipper helixes⁶², streptavidin⁶³, barnase-barstar modules⁶⁴ or the B-subunit of Escherichia coli verotoxin that self-assemble into a pentameric form⁶⁵ and can be further engineered to become decavalent⁶⁶. These antibody architectures face different challenges for their successful development as therapeutic agents. Multimeric antibody formats that rely on variable fragments (Fv) of antibodies are often associated with low stability and consequently high propensity to aggregate⁶⁷. Furthermore, dissociation of the dimerization module dictated by the affinity constant of the complex can limit the in vivo long-term stability of the molecule. In addition, maximum of 3-5 valency is usually achieved with the majority of the above-mentioned antibody formats, therefore precluding combination of high avidity and multi-specificity.

Described herein is a MULTi-specific, multi-Affinity antiBODY (multabody) platform that, in aspects, uses the apoferritin protomer as a modular subunit for the multimerization of up to 32 antibody fragments (fragment antigen binding [Fab] and fragment crystallizable [Fc]) in a single molecule. Using this approach, we have efficiently combined 4 different specificities into one single molecule including the Fab moieties of three of the best bNAbs against HIV-1 and the crystallizable fragment (Fc) from IgG1 in order to endow the molecule with multi-specificity, high avidity as well as effector functions and extended serum half-life. The resulting multabodies showed pan-virus neutralization breadth and significantly higher neutralization potency in comparison to their individual parental antibodies or to combinations of IgGs. Strikingly, the average median IC50 value of the multabodies against a 14 pseudoviruses (PsV) panel was 1 and 2 orders of magnitude lower in mass and molarity, respectively in comparison to anti-HIV trispecific N6/PGDM1400x10E8 antibody or cocktails made of the best currently know bNAbs. The multabody design described herein represents a robust and powerful plug-and-play platform to multimerize antibodies in order to enhance their therapeutic properties to suppress HIV-1 infection.

### Materials and Methods

Expression and purification of Fab-only apoferritin-based multabodies. Genes encoding the light chain of human apoferritin and the scFab-human apoferritin fusions were synthesized and cloned by GeneArt (Life Technologies) into the pHLsec expression vector. 200 ml of HEK293F cells (Thermo Fisher Scientifics) were seeded at a density of 0.8×10⁶ cells/mL in Freestyle expression media and incubated with 125 rpm oscillation at 37° C., 8% CO₂, and 70% humidity in a Multitron Pro shaker (Infors H T). Within 24 h after seeding, cells were transiently transfected using 50 μg of filtered DNA preincubated for 10 min at room temperature (RT) with the transfection reagent FectoPRO (Polyplus Transfections) in a 1:1 ratio. Plasmids encoding for scFab-human apoerritin and human apoerritin were mixed in a ratio of 1:4, 1:1, 4:1 and 1:0 in order to obtain 20%, 50%, 80% and 100% scFab valency nanoparticles, respectively. After 6-7 days, cell suspensions were harvested by centrifugation at 5000×g for 15 min and the supernatants filtered through a 0.22 μm Steritop filter (EMD Millipore). The nanoparticles were purified by affinity chromatography to the Fab and eluting after a wash. Fractions containing protein were pooled, concentrated and loaded onto a Superose 6 10/300 GL size exclusion column (GE Heathcare) in 20 mM sodium phosphate pH 8.0, 150 mM NaCl.

Design, expression and purification of 32-N and 32-I multabodies. Genes encoding for scFab and scFc fragments linked to half ferritin were generated by deletion of residues 1 to 95 (C_Ferritin) and 95 to 175 (N-Ferritin) of the light chain of human apoferritin using the KOD-Plus mutagenesis kit (Toyobo, Osaka, Japan). Furthermore, protein L binding specificity for iMab-C-Ferritin was disrupted by site directed mutagenesis through mutation of alanine 12 of the antibody light chain to a proline residue using the same mutagenesis kit. Transient transfection of the 32-N multabodies in HEK 293F cells were obtained by mixing 66 μg of the plasmids PGDM1400 scFab-human apoferritin: Fc-human apoferritin: N49P7 scFab-C-Ferritin: 10E8 scFab-C-Ferritin in a 4:2:1:1 ratio. In the case of the 32-I multabody, the plasmid N49P7 scFab-C-Ferritin was substituted by iMab scFab-C-Ferritin. The DNA mixture was filtered and incubated at RT with 60 μl of FectoPRO before adding to the cell culture. Multabodies were purified by affinity chromatography using first a HiTrap Protein A HP column (GE Healthcare) with 20 mM Tris pH 8.0, 3 M MgCl₂ and 10% glycerol elution buffer. After buffer exchange using a PD-10 desalting column (GE Healthcare), multabodies were further purified by a second affinity chromatography using a HiTrap Protein L column (GE Healthcare). Fractions containing the protein were concentrated and further purified by gel filtration on a Superose 6 10/300 GL column (GE Healthcare).

Negative-stain electron microscopy. 3 μL of multabody at a concentration approximately of 0.02 mg/mL was added to a carbon-coated copper grid for 30 s and stained with 3 μl of 2% uranyl formate. Staining excess was immediately removed from the grid using Whatman No. 1 filter paper and an additional 3 μl of 2% uranyl formate was added for 20 s. Grids were imaged using a field-emission FEI Tecnai F20 electron microscope operating at 200 kV and equipped with an Orius charge-coupled device (CCD) camera (Gatan Inc.)

Biolayer interferometry. Binding kinetics measurements were conducted using an Octet RED96 BLI system (Pall ForteBio) in PBS pH 7.4, 0.01% BSA and 0.002% Tween. A unique His-tagged ligand for each of the multabody components was selected and loaded onto Ni-NTA biosensors to reach a signal response of 0.8 nm. Association rates were measured by transferring the loaded biosensors to wells containing serial dilutions of the multabodies (50-25-12.5-6.25-3.1-1.5 nM) and buffer containing wells, respectively. Dissociation rates were measured by dipping the biosensors into buffer-containing wells. The duration of each of these two steps was 180 s. To achieve selective binding to PGDM1400, a D368R mutation in the CD4bs of the BG5050 SOSIP.664 trimer was introduced and consequently, binding of N49P7 to this antigen was disrupted. Similarly, the gp120 subunit 93TH057, MPER peptide fused to mVenus, the soluble CD4 and the hFcRn in complex with β2-microglobulin were produced as the only ligands for N49P7, 10E8, iMab and Fc respectively. The capacity of the multabodies to undergo endosomal recycling was tested by measuring their binding to the hFcRn β2-microglobulin complex at physiological (7.5) and acidic (5.6) pH.

Size-exclusion chromatography in-line with multi-angle light scattering (SEC-MALS). A MiniDAWN TREOS and an Optilab T-rEX refractometer (Wyatt) were used in-line to an Agilent Technologies 1260 infinity II HPLC. 50 μg of 24-mer PGDM1400 scFab multabody, multabody 32-N and multabody 32-I were loaded onto a Superose 6 10/300 (GE Healthcare) column in 20 mM sodium phosphate pH 8.0, 150 mM NaCl. Data collection and analysis were performed using the ASTRA software (Wyatt).

Melting and aggregation temperature measurements. The melting temperature (Tₘ) and aggregation temperature (T_{agg}) of the multabodies, parental IgGs, the 12-mer homo-oligomeric Fabs and Fc and the N6/PGDM1400x10E8 trispecific antibody was determined using a UNit system (Unchained Labs). Tₘ was obtained by measuring the barycentric mean fluorescence while T_{agg} was determined as the temperature at which 50% increase in the static light scattering at a 266 nm wavelength relative to baseline was observed. Samples were concentrated to 1.0 mg/mL and subjected to a thermal ramp from 25 to 95° C. with 1° C. increments. The average and the standard error of 3 independent measurements were calculated using the UNit analysis software.

Virus production and TZM-bl neutralization assays. A panel of 14 HIV-1 pseudotyped viruses was generated by co-transfection of 293T cells with the HIV-1 subtype B backbone NL4-3.Luc.R⁻E plasmid (AIDS Research and Reference Reagent Program (ARRRP)) and the plasmid encoding the full-length Env clone, as previously described⁷³. HIV isolates X2988, ZM106.9 and 3817 were kindly provided by the collaboration for AIDS Vaccine Discovery (CAVD), SF162 from J. L. Nieva. (Biofisika Institute) and pCNE8, 1632, THRO, 278, ZM197, JRCSF, t257, Du422 and BG505 from NIH ARRRP. Neutralization was determined in a single-cycle neutralization assay using the standard TZM-bl neutralization assay. Briefly, antibodies and antibody-based particles were incubated with a 10-15% tissue culture infectious dose of pseudovirus for 1 h at 37° C. prior to a 44-72 h incubation with TZM-bl cells. Virus neutralization was monitored by adding Britelite plus reagent (PerkinElmer) to the cells and measuring luminescence in relative light units (RLUs) using a Synergy Neo2 Multi-Mode Assay Microplate Reader (Biotek Instruments).

Pharmacokinetics and immunogenicity studies. In vivo studies were performed using 20 g C57BL/6 male mice. A surrogate multabody composed of the scFab and scFc fragments of the mouse HD37 IgG2a fused to the N-terminus of the light chain of mouse apoferritin was used for the study. HD37 scFab-mFerritin: Fc-mFerritin: mFerritin in a 2:1:1 ratio was transfected and purified following the procedure described above. L35A, L234A and P329G mutations were introduced in the mouse IgG2a Fc-construct to silence effector functions of the multabody⁷⁴. A single injection of 5 mg/kg of the multabodies or the control samples (HD37 IgG1, HD37 IgG2a and hpFerritin-PfCSP malaria peptide) in 200 μL of PBS (pH 7.5) were subcutaneously injected. Blood samples were collected at multiple time points and serum samples were assessed for levels of circulating antibodies and ADA by ELISA. Briefly, 96-well Pierce Nickel Coated Plates (Thermo Fisher) were coated with 50 μL at 0.5 μg/ml of the His₆ₓ-tagged antigen hCD19 to determine circulating HD37-specific concentrations using reagent-specific standard curves for IgGs and multabodies. For anti-drug antibody determination, Nunc MaxiSorp plates (Biolegend) were coated with a 12-mer HD37 scFab multabody or with the hpFerritin PfCSP malaria peptide. HRP-ProteinA (Invitrogen) was used as a secondary molecule and the chemiluminescence signal was quantified using a Synergy Neo2 Multi-Mode Assay Microplate Reader (Biotek Instruments).

### Results

Multabodies can neutralize HIV-1 up to 500 times more potently than gold-standard IgGs. The strong self-assembly properties of the light chain of human apoferritin was used to multimerize Fabs onto the surface of a hollow spherical protein cage formed by 24 monomers. Indeed, apoferritin self-assembles into a 12 nm diameter structure composed of 24 identical polypeptides and is readily amenable to genetic fusions⁷⁰. The N-terminus of each apoferritin subunit points outwards of the spherical cage and it is therefore accessible for the genetic fusion of proteins of interest. In order to maintain all properties of an IgG molecule including high-thermostability and correct chain pairing, we generated apoferritin fusions to single-chain Fab (scFab) and single-chain Fc (scFc) fragments. Upon folding, apoferritin subunits act as building blocks that drive the multimerization of the 24 proteins fused to its N-terminus (FIG. 1). Importantly, presentation of multiple specificities on the same molecule (e.g. Fabs and Fc) can be achieved and controlled by co-transfection of selected ratios of the DNA encoding each of the components followed by stringent affinity purification protocols selecting for all specificities (e.g., Protein L combined with Protein A affinity chromatography select for the Fab and the Fc, respectively).

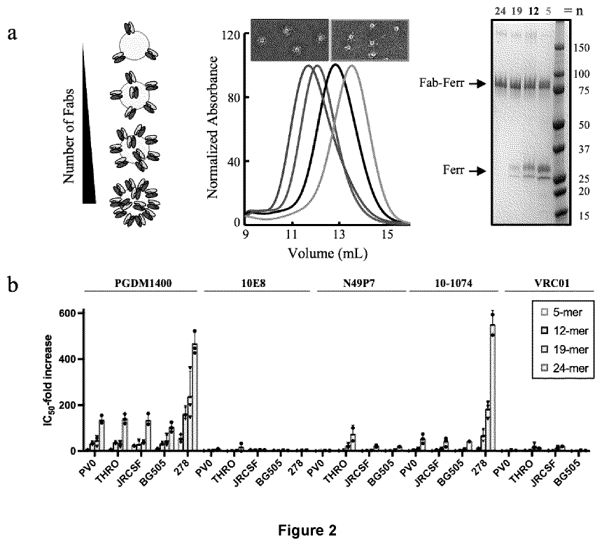

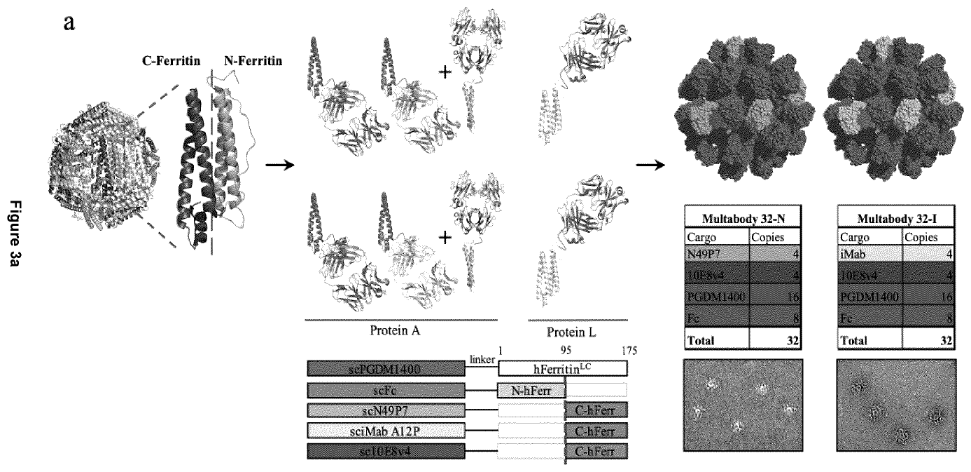

First, we investigated the impact of multi-valency for HIV-1 bNAbs displayed on our novel multabody platform in their ability to block virus infection, and compared it to the standard bi-valent IgG display for same bNAbs. A panel of bNAbs with different specificities towards Env were selected and their scFabs were multimerized at different densities by co-transfection of scFab-human apoferritin-encoding plasmids together with different ratios of unconjugated apoferritin (FIG. 2a). Multabodies assembled into monodisperse, well-formed spherical particles with an uninterrupted ring of density and regularly spaced protruding Fabs (FIG. 2a). Strikingly, PGDM1400, the most potent anti-HIV bNAb described to date (median IC50 value=0.003 μg/mL) showed 100 to 500-fold higher neutralization (fold improvement in median IC₅₀ (nM) value) as a 24-mer multabody compared to its IgG format against a five-pseudoviruses panel (FIG. 2b). bNAb 10-1074 also showed a drastic improvement in neutralization potency as a multabody compared to its IgG, whereas bNAbs 10E8, N49P7 and VRC01, while still effective, did not show the same enhancement.

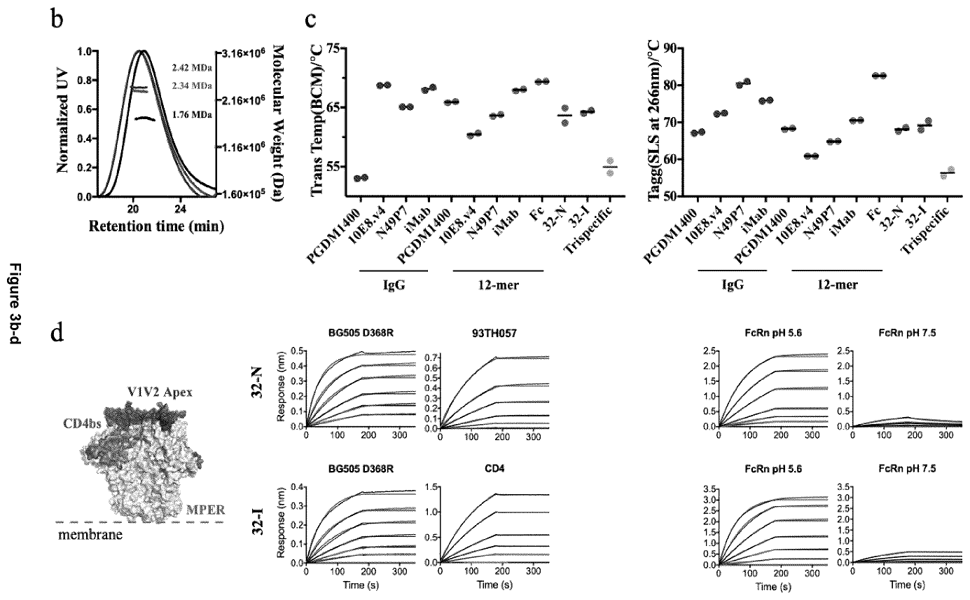

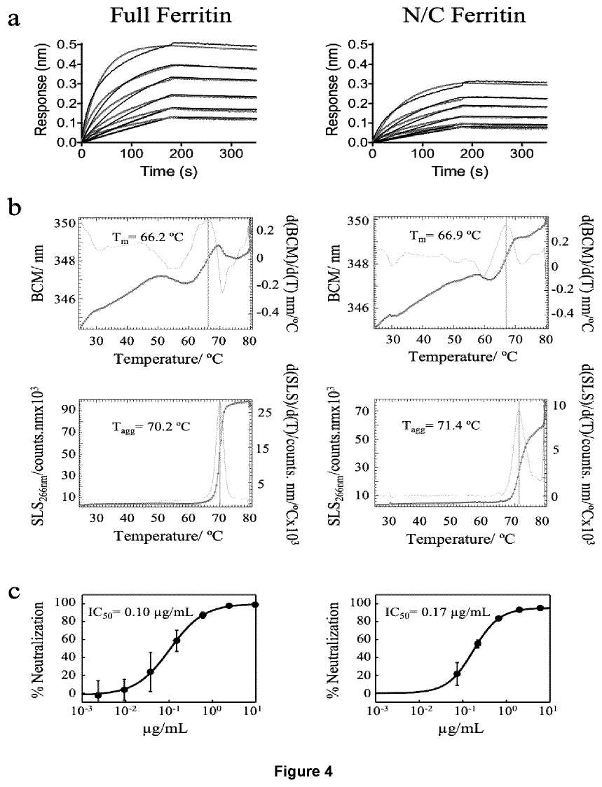

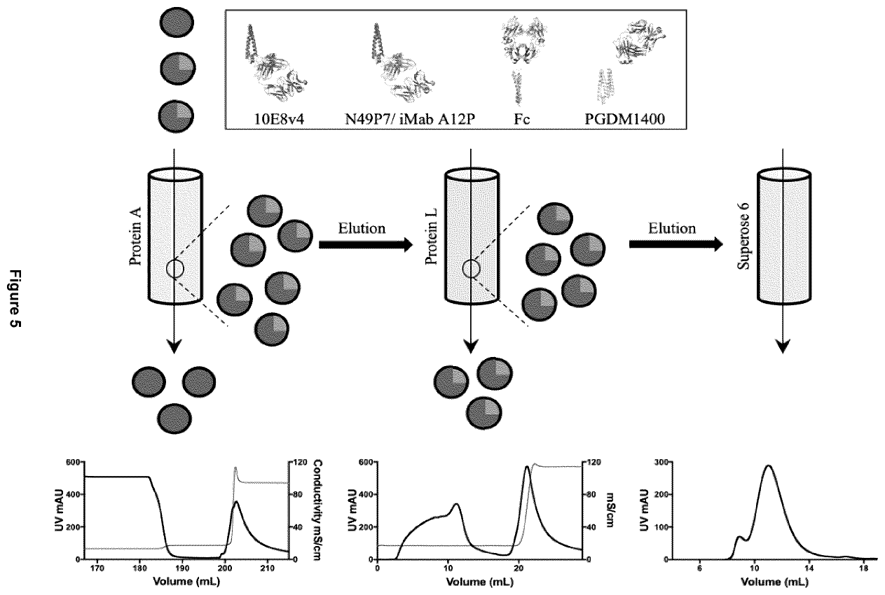

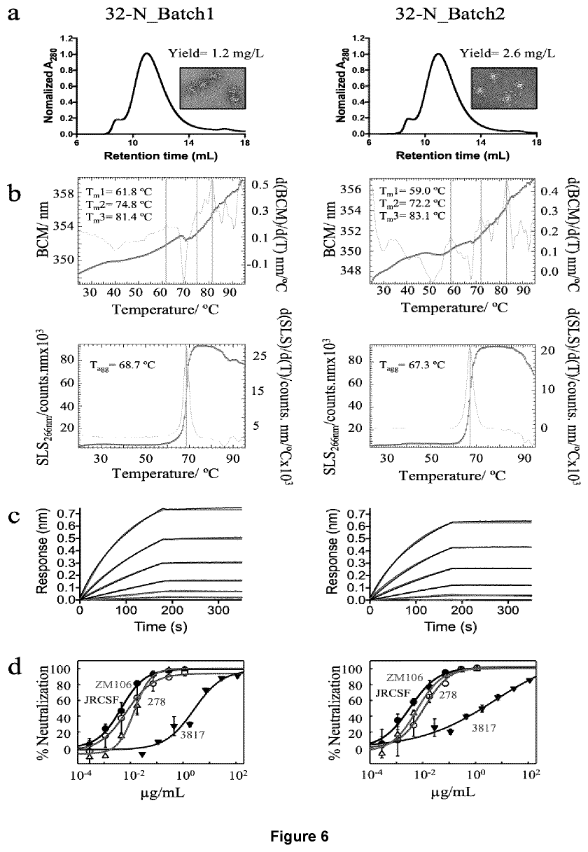

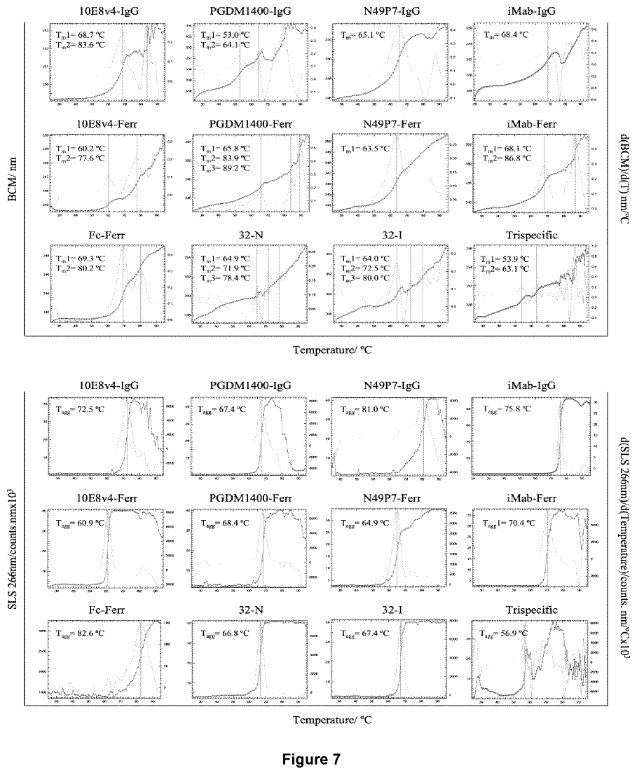

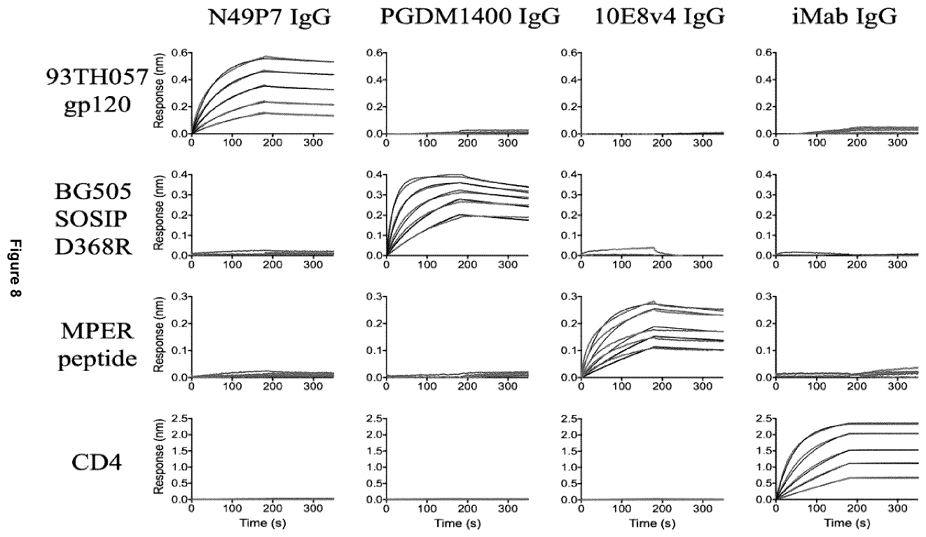

Apoferritin engineering results in efficient hetero-oligomerization of 32-mer multabodies. Next, we sought to improve the breadth of the exceptionally potent 24-mer PGDM1400 multabody by conferring multi-specificity to the molecule. For this purpose, we combined PGDM1400 Fabs with the Fabs of near-pan neutralizing antibodies 10E8v4 (a modified 10E8 with improved solubility⁷¹) and N49P7, in addition to the Fc fragment of the human IgG1 isotype. To achieve this level of hetero-oligomerization of four components (three Fabs and one Fc), we split the human apoferritin structure into two subunits (N-Ferritin and C-Ferritin) and attached Fabs at the N-terminus of each half (FIG. 3a). Split apoferritin complementation led to self-association of the two halves and consequently resulted in a very efficient hetero-dimerization process of the fused proteins. Importantly, no significant differences were observed between the biophysical and functional properties of multabodies assembled with split and full-length apoferritin (FIG. 4). This design allowed a simple two-step purification procedure to select for multabodies with four different specificities (FIG. 5) and showed high batch-to-batch homogeneity (FIG. 6). Furthermore, the bipartite split of apoferritin allowed to accommodate additional Fab/Fc fragments compared to the standard apoferritin building block, up to a maximum of 32 components per molecule (FIG. 3b). The 8 extra positions (from the conventional 24 to 32 in our engineered platform) play an important role in providing multi-specificity without sacrificing much of the gain in potency observed in the 24-mer PGDM1400 multabody. As such, multabody 32-N was designed to achieve 16 copies of PGDM1400, 8 copies of Fc, 4 copies of 10E8v4, and 4 copies of N49P7 by co-transfection of the scFab- and scFc-encoding plasmids in a 4:2:1:1 ratio, respectively (FIG. 3a). Multabody 32-N formed highly-decorated and homogeneous particles (FIG. 3b), and showed transition temperatures of unfolding and aggregation similar to the Tm and Tagg distribution of the corresponding IgG molecules, and also as previously reported for IgGs⁷² (FIGS. 3c and 7). Binding kinetics experiments were used to demonstrate that each component of the multabody (PDGM1400, N49P7, 10E8v4 and the Fc fragment) was able to engage its epitope by binding epitope-specific molecules: BG505 SOSIP D368R, 93TH057 gp120, an MPER peptide and human FcRn, respectively (FIG. 3d). No individual IgG molecules had the ability to bind all antigens (FIG. 8). The ability of 32-N to engage its multiple antigens indicates that the geometry imposed by the oligomeric format of the multabody does not negatively impact antigen binding, presumably because the binding interfaces are outwardly facing.

To explore whether a multabody could also be designed that cross-targets the HIV Env and T-cell receptor CD4, we replaced N49P7 with iMab, a CD4-directed post-attachment inhibitor that has been shown to efficaciously eradicate HIV^{68,69}. The multabody containing PDGM1400, iMab, 10E8v4 and the Fc fragment (termed 32-I) showed similar homogeneity, thermostability and multi-specificity as 32-N (FIGS. 3, 7, and 8), emphasizing the robust plug-and-play nature of the multabody platform where antibody sequences can be easily swapped to alter specificities.

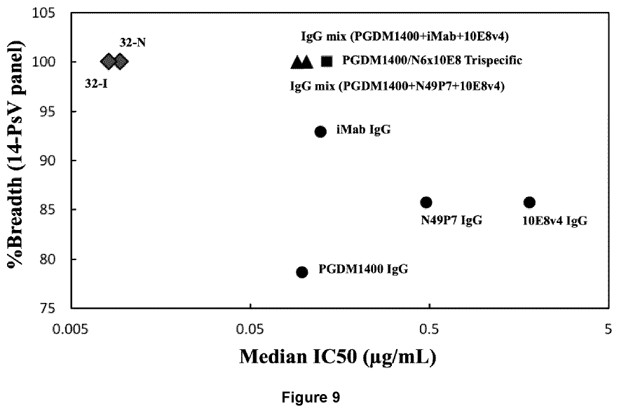

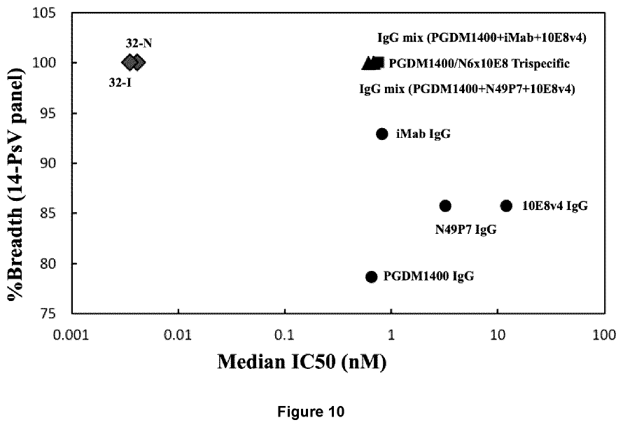

HIV-1 multabodies exhibit exceptional pan-neutralizing activity and potency. Neutralization potency and breadth of multabodies 32-N and 32-I were assessed against a panel of 14-pseudoviruses (PsVs) in a standardized in vitro TZM-bl neutralization assays⁷³. The 14-PsV panel was designed to include low-sensitivity PsVs with a minimum of one resistant PsV for each bNAb being evaluated. The IC₅₀ value and breadth of the multabodies were compared to (i) each individual IgG, (ii) an IgG cocktail that contains the same relative amount of each IgG present in the multabody and (iii) the N6/PGDM1400x10E8 trispecific antibody⁴³. 32-N and 32-I multabodies displayed 100% breadth against this panel with a median 1050 value of 0.0093 μg/mL (4 pM) and 0.0085 μg/mL (3.5 pM), respectively (FIGS. 9 and 10). Full virus coverage was also achieved by the IgG mixture and the trispecific antibody. However, the potency of the combined cocktails or trispecific against all PsVs tested was similar to that of the best mAb when tested alone (Table 1). As such, more than a 10-fold and 100-fold decrease in the median 1050 value when calculated in μg/mL and nM, respectively was obtained for the multabodies in comparison to the 1050 value of the IgG cocktails and the trispecific antibody (FIGS. 9 and 10, Tables 1 and 2).

TABLE 1

| | 10E8v4 | N49P7 | PGDM1400 | Ibalizumab | Mix | Mix | | | | |
|---|---|---|---|---|---|---|---|---|---|---|
| | IgG | IgG | IgG | IgG | (N49P7) | (Ibalizumab) | Trispecific | 32-N | 32-I | μg/mL |
| pCNE8 | 0.96 | 0.28 | 0.0027 | 0.062 | 0.0093 | 0.015 | 0.024 | 0.0032 | 0.0013 | <0.005 |
| X1632 | 2.02 | >50 | 0.018 | >50 | 0.034 | 0.036 | 0.03 | 0.0045 | 0.008 | 0.005-0.01 |
| THRO | 0.37 | 7.5 | 0.09 | 0.14 | 0.15 | 0.17 | 0.31 | 0.058 | 0.033 | 0.01-0.1 |
| 278 | 1.8 | >50 | 0.16 | 0.67 | 0.15 | 0.14 | 1.2 | 0.009 | 0.008 | 0.1-1 |
| 3817 | 3.1 | 3.3 | >50 | 0.18 | 7 | 0.31 | 1.2 | 9.1 | 1.8 | 1-50 |
| ZM197 | 0.19 | 0.58 | 0.11 | 0.042 | 0.037 | 0.062 | 0.073 | 0.0096 | 0.0044 | >50 |
| JRCSF | 1.8 | 0.12 | 0.0074 | 0.064 | 0.0064 | 0.014 | 0.026 | 0.0054 | 0.004 | |
| T257 | 1.5 | 1.2 | 0.012 | 0.095 | 0.0082 | 0.0078 | 0.028 | 0.0037 | 0.0015 | |
| ZM106 | >50 | 0.18 | 0.013 | 0.2 | 0.013 | 0.014 | 0.017 | 0.0089 | 0.0031 | |
| X2088 | >50 | 0.38 | >50 | 0.13 | 2 | 0.66 | 0.56 | 5.2 | 3.1 | |
| PV0 | 6.3 | 0.28 | 1.3 | 0.11 | 0.76 | 0.4 | 1.3 | 0.33 | 0.12 | |
| Du422 | 0.55 | 10 | 6 | 0.079 | 0.25 | 0.31 | 0.19 | 2.5 | 0.7 | |
| BG505 | 1.4 | 0.21 | 0.0032 | 0.12 | 0.0081 | 0.013 | 0.059 | 0.0045 | 0.0026 | |
| SF162 | 3 | 0.15 | >50 | 7.5 | 0.56 | 1.4 | 0.33 | 1.3 | 2.5 | |
| Median | 1.8 | 0.48 | 0.098 | 0.12 | 0.091 | 0.1 | 0.13 | 0.0093 | 0.008 | |

TABLE 2

| | 10E8v4 | N49P7 | PGDM1400 | Ibalizumab | Mix | Mix | | | | |
|---|---|---|---|---|---|---|---|---|---|---|
| | IgG | IgG | IgG | IgG | (N49P7) | (Ibalizumab) | Trispecific | 32-N | 32-I | nM |
| pCNE8 | 6.4 | 1.8 | 0.018 | 0.41 | 0.062 | 0.1 | 0.14 | 0.0014 | 0.00057 | <0.005 |
| X1632 | 13 | >100 | 0.12 | >100 | 0.22 | 0.24 | 0.17 | 0.002 | 0.0034 | 0.005-0.01 |
| THRO | 2.5 | 50 | 0.61 | 0.96 | 0.97 | 1.1 | 1.7 | 0.025 | 0.014 | 0.01-0.1 |
| 278 | 12 | >100 | 1.1 | 4.5 | 1 | 0.95 | 7.1 | 0.0039 | 0.0035 | 0.1-1 |
| 3817 | 21 | 22 | >100 | 1.2 | 47 | 2.1 | 6.7 | 3.9 | 0.79 | 1-50 |
| ZM197 | 1.3 | 3.9 | 0.68 | 0.28 | 0.24 | 0.41 | 0.42 | 0.0042 | 0.0019 | >50 |
| JRCSF | 12 | 0.85 | 0.049 | 0.43 | 0.042 | 0.096 | 0.15 | 0.0023 | 0.0017 | |
| T257 | 9.8 | 8.3 | 0.08 | 0.63 | 0.054 | 0.052 | 0.16 | 0.0016 | 0.00068 | |
| ZM106 | >100 | 1.2 | 0.084 | 1.3 | 0.09 | 0.093 | 0.1 | 0.0039 | 0.0014 | |
| X2088 | >100 | 2.5 | >100 | 0.84 | 13 | 4.4 | 3.2 | 2.3 | 1.3 | |
| PV0 | 42 | 1.9 | 8.4 | 0.75 | 5 | 2.6 | 7.2 | 0.15 | 0.052 | |
| Du422 | 3.7 | 66 | 40 | 0.53 | 1.7 | 2.1 | 1.1 | 1.08 | 0.31 | |
| BG505 | 9 | 1.4 | 0.02 | 0.8 | 0.054 | 0.085 | 0.34 | 0.0019 | 0.0011 | |
| SF162 | 20 | 1 | >100 | 50 | 3.7 | 9.1 | 1.9 | 0.58 | 1.1 | |
| Median | 12 | 3.2 | 0.65 | 0.8 | 0.61 | 0.68 | 0.77 | 0.00405 | 0.00345 | |

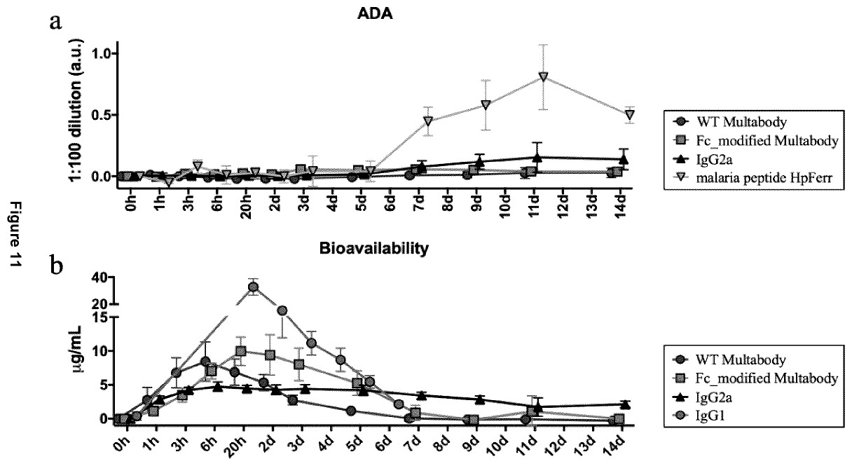

In vivo pharmacokinetics and anti-drug antibody profile of multabodies are similar to corresponding IgG. We next examined the in vivo toxicity, immunogenicity and bioavailability of a multabody after a subcutaneous administration of 5 mg/kg in mice. To assess our novel platform technology, we used a species-matched surrogate multabody that consists of a mouse Fab and a mouse Fc (IgG2a isotype) fused to the mouse apoferritin subunit, in contrast to the all-human components used for the HIV-1 multabodies targeted for use in humans. The Fab specificity that was selected for this surrogate multabody is one that does not bind an endogenous mouse protein, analogous to a HIV-1 human mAb that would not bind an endogenous human protein. Multabody administration was well tolerated with no decrease in body weight or visible signs of toxicity. The surrogate multabody did not induce a significant immunogenic response in mice; levels of antidrug-antibodies (ADA) detected after 14 days were negligible for both the surrogate multabody and its sequenced-matched IgG2a (FIG. 11b). This is in contrast to a highly immunogenic particle that displays the malaria circumsporozoite protein (CSP) on the surface of Helicobacter Pylori ferritin (hpFerritin) that was used as positive control of immunogenicity. In addition, the surrogate multabody showed days of in vivo exposure in a similar range to parental IgG1 and IgG2a molecules (FIG. 11b). Serum half-life could be extended by introduction of the LALAP mutations reported to silence the Fc effector function⁷⁴. Overall, these data demonstrate the feasibility to tune the bioavailability properties of our multabody platform and provide an encouraging set of initial in vivo validation for its developability.

### REFERENCES

1. Conley, A. J. et al. Neutralization of divergent human immunodeficiency virus type 1 variants and primary isolates by IAM-41-2F5, an anti-gp41 human monoclonal antibody. Proc. Natl. Acad. Sci. (2006). doi:10.1073/pnas.91.8.3348
2. Stiegler, G. et al. A Potent Cross-Clade Neutralizing Human Monoclonal Antibody against a Novel Epitope on gp41 of Human Immunodeficiency Virus Type 1. AIDS Res. Hum. Retroviruses (2002). doi:10.1089/08892220152741450
3. Zwick, M. B. et al. Broadly Neutralizing Antibodies Targeted to the Membrane-Proximal External Region of Human Immunodeficiency Virus Type 1 Glycoprotein gp41. J. Virol. (2002). doi:10.1128/jvi.75.22.10892-10905.2001
4. BUCHACHER, A. et al. Generation of Human Monoclonal Antibodies against HIV-1 Proteins; Electrofusion and Epstein-Barr Virus Transformation for Peripheral Blood Lymphocyte Immortalization. AIDS Res. Hum. Retroviruses (2009). doi:10.1089/aid.1994.10.359
5. Barbas, C. F. et al. Recombinant human Fab fragments neutralize human type 1 immunodeficiency virus in vitro. Proc. Natl. Acad. Sci. U.S.A (1992).
6. Burton, D. R. et al. Efficient neutralization of primary isolates of HIV-1 by a recombinant human monoclonal antibody. Science (80-.). (1994). doi:10.1126/science.7973652
7. Wu, X. et al. Rational design of envelope identifies broadly neutralizing human monoclonal antibodies to HIV-1. Science (80-.). (2010). doi:10.1126/science.1187659
8. Scheid, J. F. et al. Broad diversity of neutralizing antibodies isolated from memory B cells in HIV-infected individuals. Nature (2009). doi:10.1038/nature07930
9. Sok, D. et al. A Recombinant HIV Envelope Trimer Selects for Quaternary Dependent Antibodies Targeting the Trimer Apex. AIDS Res. Hum. Retroviruses (2014). doi:10.1089/aid.2014.5002.abstract
10. Walker, L. M. et al. Broad and Potent Neutralizing Antibodies from an African Donor Reveal a New HIV-1 Vaccine Target TL—326. Science (80-.). (2009). doi:10.1126/science.1178746
11. Walker, L. M. et al. Broad neutralization coverage of HIV by multiple highly potent antibodies. Nature (2011). doi:10.1038/nature10373
12. Doria-Rose, N. A. et al. Developmental pathway for potent V1V2-directed HIV-neutralizing antibodies. Nature (2014). doi:10.1038/nature13036
13. Huang, J. et al. Broad and potent neutralization of HIV-1 by a gp41-specific human antibody. Nature (2012). doi:10.1038/nature11544
14. Sajadi, M. M. et al. Identification of Near-Pan-neutralizing Antibodies against HIV-1 by Deconvolution of Plasma Humoral Responses. Cell (2018). doi:10.1016/j.cell.2018.03.061
15. Huang, J. et al. Identification of a CD4-Binding-Site Antibody to HIV that Evolved Near-Pan Neutralization Breadth. Immunity (2016). doi:10.1016/j.immuni.2016.10.027
16. Pejchal, R. et al. A potent and broad neutralizing antibody recognizes and penetrates the HIV glycan shield. Science (80-.). (2011). doi:10.1126/science.1213256
17. Blattner, C. et al. Structural delineation of a quaternary, cleavage-dependent epitope at the gp41-gp120 interface on intact HIV-1 env trimers. Immunity (2014). doi:10.1016/j.immuni.2014.04.008
18. Mouquet, H. et al. Complex-type N-glycan recognition by potent broadly neutralizing HIV antibodies. Proc. Natl. Acad. Sci. (2012). doi:10.1073/pnas.1217207109
19. Scheid, J. F. et al. Sequence and Structural Convergence of Broad and Potent HIV Antibodies That Mimic CD4 Binding. Science (80-.). (2011). doi:10.1126/science.1207227
20. Baba, T. W. et al. Human neutralizing monoclonal antibodies of the IgG1 subtype protect against mucosal simian-human immunodeficiency virus infection. Nat. Med. (2000). doi:10.1038/72309
21. Hessell, A. J. et al. Effective, low-titer antibody protection against low-dose repeated mucosal SHIV challenge in macaques. Nat. Med. (2009). doi:10.1038/nm.1974
22. Hessell, A. J. et al. Broadly neutralizing human anti-HIV antibody 2G12 is effective in protection against mucosal SHIV challenge even at low serum neutralizing titers. PLoS Pathog. (2009). doi:10.1371/journal.ppat.1000433
23. Hofmann-Lehmann, R. et al. Postnatal pre- and postexposure passive immunization strategies: Protection of neonatal macaques against oral simian-human immunodeficiency virus challenge. J. Med. Primatol. (2002). doi:10.1034/j.1600-0684.2002.01014.x
24. Hofmann-Lehmann, R. et al. Postnatal Passive Immunization of Neonatal Macaques with a Triple Combination of Human Monoclonal Antibodies against Oral Simian-Human Immunodeficiency Virus Challenge. J. Virol. (2002). doi:10.1128/jvi.75.16.7470-7480.2001
25. van der Velden, Y. U. et al. Short Communication: Protective Efficacy of Broadly Neutralizing Antibody PGDM1400 Against HIV-1 Challenge in Humanized Mice. AIDS Res. Hum. Retroviruses (2018). doi:10.1089/aid.2018.0114
26. Klein, F. et al. HIV therapy by a combination of broadly neutralizing antibodies in humanized mice. Nature (2012). doi:10.1038/nature11604
27. Deruaz, M. et al. Protection of humanized mice from repeated intravaginal HIV challenge by passive immunization: A model for studying the efficacy of neutralizing antibodies in vivo. J. Infect. Dis. (2016). doi:10.1093/infdis/jiw203
28. Horwitz, J. A. et al. HIV-1 suppression and durable control by combining single broadly neutralizing antibodies and antiretroviral drugs in humanized mice. Proc. Natl. Acad. Sci. (2013). doi:10.1073/pnas.1315295110
29. Mehandru, S. et al. Adjunctive Passive Immunotherapy in Human Immunodeficiency Virus Type 1-Infected Individuals Treated with Antiviral Therapy during Acute and Early Infection. J. Virol. (2007). doi:10.1128/jvi.01340-07
30. Caskey, M. et al. Viraemia suppressed in HIV-1-infected humans by broadly neutralizing antibody 3BNC117. Nature (2015). doi:10.1038/nature14411
31. Caskey, M. et al. Antibody 10-1074 suppresses viremia in HIV-1-infected individuals. Nat. Med. (2017). doi:10.1038/nm.4268
32. Lynch, R. M. et al. Virologic effects of broadly neutralizing antibody VRC01 administration during chronic HIV-1 infection. Sci. Transl. Med. (2015). doi:10.1126/scitranslmed.aad5752
33. Ledgerwood, J. E. et al. Safety, pharmacokinetics and neutralization of the broadly neutralizing HIV-1 human monoclonal antibody VRC01 in healthy adults. Clin. Exp. Immunol. (2015). doi:10.1111/cei.12692
34. Toma, J. et al. Loss of Asparagine-Linked Glycosylation Sites in Variable Region 5 of Human Immunodeficiency Virus Type 1 Envelope Is Associated with Resistance to CD4 Antibody Ibalizumab. J. Virol. (2011). doi:10.1128/jvi.02237-10
35. Jacobson, J. M. et al. Antiviral Activity of Single-Dose PRO 140, a CCR5 Monoclonal Antibody, in HIV-Infected Adults. J. Infect. Dis. (2008). doi:10.1086/592169
36. Onafuwa-Nuga, A. & Telesnitsky, A. The Remarkable Frequency of Human Immunodeficiency Virus Type 1 Genetic Recombination. Microbiol. Mol. Biol. Rev. (2009). doi:10.1128/mmbr.00012-09
37. Sather, D. N. et al. Broadly Neutralizing Antibodies Developed by an HIV-Positive Elite Neutralizer Exact a Replication Fitness Cost on the Contemporaneous Virus. J. Virol. (2012). doi:10.1128/JVI.01893-12
38. Lynch, R. M. et al. HIV-1 Fitness Cost Associated with Escape from the VRC01 Class of CD4 Binding Site Neutralizing Antibodies. J. Virol. (2015). doi:10.1128/jvi.03608-14
39. Pietzsch, J. et al. Human anti-HIV-neutralizing antibodies frequently target a conserved epitope essential for viral fitness. J. Exp. Med. (2010). doi:10.1084/jem.20101176
40. Asokan, M. et al. Bispecific Antibodies Targeting Different Epitopes on the HIV-1 Envelope Exhibit Broad and Potent Neutralization. J. Virol. (2015). doi:10.1128/jvi.02097-15
41. Bournazos, S., Gazumyan, A., Seaman, M. S., Nussenzweig, M. C. & Ravetch, J. V. Bispecific Anti-HIV-1 Antibodies with Enhanced Breadth and Potency. Cell (2016). doi:10.1016/j.cell.2016.04.050
42. Huang, Y. et al. Engineered Bispecific Antibodies with Exquisite HIV-1-Neutralizing Activity. Cell (2016). doi:10.1016/j.cell.2016.05.024
43. Xu, L. et al. Trispecific broadly neutralizing HIV antibodies mediate potent SHIV protection in macaques. Science (80-.). (2017). doi:10.1126/science.aan8630
44. Steinhardt, J. J. et al. Rational design of a trispecific antibody targeting the HIV-1 Env with elevated anti-viral activity. Nat. Commun. (2018). doi:10.1038/s41467-018-03335-4
45. Khan, S. N. et al. Targeting the HIV-1 Spike and Coreceptor with Bi- and Trispecific Antibodies for Single-Component Broad Inhibition of Entry. J. Virol. (2018). doi:10.1128/jvi.00384-18
46. Rudicell, R. S. et al. Enhanced Potency of a Broadly Neutralizing HIV-1 Antibody In Vitro Improves Protection against Lentiviral Infection In Vivo. J. Virol. (2014). doi:10.1128/JVI.02213-14
47. Kwon, Y. D. et al. Surface-Matrix Screening Identifies Semi-specific Interactions that Improve Potency of a Near Pan-reactive HIV-1-Neutralizing Antibody. Cell Rep. (2018). doi:10.1016/j.celrep.2018.01.023
48. Rujas, E. et al. Functional Optimization of Broadly Neutralizing HIV-1 Antibody 10E8 by Promotion of Membrane Interactions. J. Virol. (2018). doi:10.1128/jvi.02249-17
49. Diskin, R. et al. Increasing the potency and breadth of an HIV antibody by using structure-based rational design. Science (80-.). (2011). doi:10.1126/science.1213782
50. Wu, H. et al. Ultra-potent antibodies against respiratory syncytial virus: Effects of binding kinetics and binding valence on viral neutralization. J. Mol. Biol. (2005). doi:10.1016/j.jmb.2005.04.049
51. Icenogle, J. et al. Neutralization of poliovirus by a monoclonal antibody: Kinetics and stoichiometry. Virology (1983). doi:10.1016/0042-6822(83) 90154-X
52. Cavacini, L. A., Emes, C. L., Power, J., Duval, M. & Posner, M. R. Effect of antibody valency on interaction with cell-surface expressed HIV-1 and viral neutralization. J. Immunol. (1994).
53. Smith, R. I., Coloma, M. J. & Morrison, S. L. Addition of a mu-tailpiece to IgG results in polymeric antibodies with enhanced effector functions including complement-mediated cytolysis by IgG4. J. Immunol. (1995).
54. Olafsen, T., Rasmussen, B. I., Norderhaug, L., Bruland, Ø. S. & Sandlie, I. IgM secretory tailpiece drives multimerisation of bivalent scFv fragments in eukaryotic cells. Immunotechnology (1998). doi:10.1016/51380-2933(98) 00014-1
55. Miller, K. et al. Design, Construction, and In Vitro Analyses of Multivalent Antibodies. J. Immunol. (2003). doi:10.4049/jimmunol.170.9.4854
56. Kipriyanov, S. M. et al. Bispecific tandem diabody for tumor therapy with improved antigen binding and pharmacokinetics. J. Mol. Biol. (1999). doi:10.1006/jmbi.1999.3156
57. Lu, D. et al. Di-diabody: A novel tetravalent bispecific antibody molecule by design. J. Immunol. Methods (2003). doi:10.1016/S0022-1759(03) 00251-5
58. Wu, C. et al. Simultaneous targeting of multiple disease mediators by a dual-variable-domain immunoglobulin. Nat. Biotechnol. (2007). doi:10.1038/nbt1345
59. Klein, C., Schaefer, W. & Regula, J. T. The use of CrossMAb technology for the generation of bi- and multispecific antibodies. mAbs (2016).

doi:10.1080/19420862.2016.1197457
60. Steinmetz, A. et al. CODV-Ig, a universal bispecific tetravalent and multifunctional immunoglobulin format for medical applications. MAbs (2016). doi:10.1080/19420862.2016.1162932
61. Kubetzko, S., Balic, E., Waibel, R., Zangemeister-Wittke, U. & Plückthun, A. PEGylation and multimerization of the anti-p185HER-2 single chain Fv fragment 4D5: Effects on tumor targeting. J. Biol. Chem. (2006). doi:10.1074/jbc.M604127200
62. Pack, P., MÜller, K., Zahn, R. & Plückthun, A. Tetravalent Miniantibodies with High Avidity Assembling in Escherichia colo. Journal of Molecular Biology (1995). doi:10.1006/jmbi.1994.0062
63. Kipriyanov, S. M. et al. Affinity enhancement of a recombinant antibody: formation of complexes with multiple valency by a single-chain Fv fragment-core streptavidin fusion. “Protein Eng. Des. Sel. (2007). doi:10.1093/protein/9.2.203
64. Deyev, S. M., Waibel, R., Lebedenko, E. N., Schubiger, A. P. & Plückthun, A. Design of multivalent complexes using the barnasebarstar module. Nat. Biotechnol. (2003). doi:10.1038/nbt916
65. Zhang, J. et al. Pentamerization of single-domain antibodies from phage libraries: A novel strategy for the rapid generation of high-avidity antibody reagents. J. Mol. Biol. (2004). doi:10.1016/j.jmb.2003.09.034
66. Stone, E. et al. The assembly of single domain antibodies into bispecific decavalent molecules. J. Immunol. Methods (2007). doi:10.1016/j.jim.2006.10.006
67. Worn, A. & Plückthun, A. Different equilibrium stability behavior of scFv fragments: Identification, classification, and improvement by protein engineering. Biochemistry (1999). doi:10.1021/bi9902079
68. Jacobson, J. M. et al. Safety, pharmacokinetics, and antiretroviral activity of multiple doses of ibalizumab (formerly TNX-355), an anti-CD4 monoclonal antibody, in human immunodeficiency virus type 1-infected adults. Antimicrob. Agents Chemother. (2009). doi:10.1128/AAC.00942-08
69. Kuritzkes, D. R. et al. Antiretroviral Activity of the Anti-CD4 Monoclonal Antibody TNX-355 in Patients Infected with HIV Type 1. J. Infect. Dis. (2004). doi:10.1086/380802
70. Lawson, D. M. et al. Solving the structure of human H ferritin by genetically engineering intermolecular crystal contacts. Nature (1991). doi:10.1038/349541a0
71. Kwon, Y. D. et al. Optimization of the Solubility of HIV-1-Neutralizing Antibody 10E8 through Somatic Variation and Structure-Based Design. J. Virol. (2016). doi:10.1128/jvi.03246-15
72. Goto, Y., Ichimura, N. & Hamaguchi, K. Effects of Ammonium Sulfate on the Unfolding and Refolding of the Variable and Constant Fragments of an Immunoglobulin Light Chain. Biochemistry (1988). doi:10.1021/bi00405a043
73. Montefiori, D. C. Measuring HIV neutralization in a luciferase reporter gene assay. Methods Mol. Biol. (2009). doi:10.1007/978-1-59745-170-3_26
74. Schlothauer, T. et al. Novel human IgG1 and IgG4 Fc-engineered antibodies with completely abolished immune effector functions. Protein Eng. Des. Sel. (2016). doi:10.1093/protein/gzw040
75. Graille, M. et al. Complex between Peptostreptococcus magnus protein L and a human antibody reveals structural convergence in the interaction modes of Fab binding proteins. Structure (2001). doi:10.1016/S0969-2126(01) 00630-X

1. A fusion protein comprising:
a first nanocage monomer subunit of a nanocage monomer; and
a bioactive moiety linked to the first nanocage monomer subunit;
wherein the fusion protein self-assembles with a protein comprising a second nanocage monomer subunit to form a nanocage monomer.

2. (canceled)

3. The fusion protein of claim 1, wherein the bioactive moiety comprises an antibody or fragment thereof, an antigen, a detectable moiety, a pharmaceutical agent, a diagnostic agent, or combinations thereof.

4. The fusion protein of claim 3, wherein the antibody or fragment thereof comprises an Fc fragment.

5. (canceled)

6. The fusion protein of claim 4, wherein the Fc fragment comprises one or more mutations that modulate the half-life of the fusion protein.

7. The fusion protein of claim 3, wherein the antibody or fragment thereof comprises a Fab fragment.

8-23. (canceled)

24. The fusion protein of claim 1, wherein the nanocage monomer is selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof.

25. The fusion protein of claim 24, wherein the nanocage monomer is apoferritin.

26. The fusion protein of claim 25, wherein the first and second nanocage monomer subunits interchangeably comprise the “N” and “C” regions of apoferritin.

27-34. (canceled)

35. A pair of the fusion proteins of claim 1, wherein the pair self-assembles to form a nanocage monomer, wherein the first and second nanocage monomer subunits are fused to different bioactive moieties.

36. A nanocage comprising at least one fusion protein of claim 1 and at least one second nanocage monomer subunit that self-assembles with the fusion protein to form a nanocage monomer.

37-46. (canceled)

47. The nanocage of claim 36, comprising
(1) a first fusion protein comprising:
an N- or C-ferritin, and
an antibody fragment comprising an Fc fragment, the antibody fragment linked to the N- or C-ferritin;
(2) a second fusion protein comprising:
an N- or C-ferritin, and
N49P7 Fab or iMab A12P Fab linked to the N- or C-ferritin; and
(3) a third fusion protein comprising:
an N- or C-ferritin, and 10E8v4 Fab linked to the N- or C-ferritin; and
(4) at least one whole ferritin, optionally fused to a bioactive moiety that is different from the antibody fragment comprising an Fc fragment, the N49P7 Fab or iMab A12P Fab, or the 10E8v4 Fab;
wherein at least one of the first, second, and third fusion proteins comprises N-ferritin, and at least one the first, second, and third fusion proteins comprises C-ferritin.

48-60. (canceled)

61. A therapeutic or prophylactic composition or vaccine comprising the nanocage of claim 36.

62. A nucleic acid molecule encoding the fusion protein of claim 1.

63. A vector comprising the nucleic acid molecule of claim 62.

64. A host cell comprising the vector of claim 63 and producing the fusion protein encoded by the nucleic acid molecule.

65. (canceled)

66. A method for immunizing a subject, treating a disease or condition, and/or preventing a disease or condition, the method comprising administering the nanocage of claim 36.

67. (canceled)

68. A method for diagnostic imaging, the method comprising administering the nanocage of claim 36 to a subject, tissue, or sample, wherein the nanocage comprises a diagnostic label, such as a fluorescent protein or magnetic imaging moiety, and imaging the subject, tissue, or sample.

69-78. (canceled)

79. A nanocage comprising a plurality of fusion proteins,
wherein each fusion protein comprises a ferritin light chain and an Fab fragment,
wherein the Fab fragment is capable of specifically binding to an antigen,
wherein the Fab fragment of each fusion protein is exposed on the nanocage's exterior, and
wherein the plurality comprises at least 12 fusion proteins.

80-91. (canceled)

92. A nanocage comprising a plurality of first fusion proteins and a plurality of second fusion proteins,
wherein each first fusion protein comprises a nanocage monomer or a subunit thereof and an Fab fragment capable of specifically binding to an antigen, and
wherein each second fusion protein comprises a nanocage monomer or a subunit thereof and an Fc fragment.

93-96. (canceled)

97. A nanocage comprising a plurality of first fusion proteins and a plurality of second fusion proteins, wherein
(a) (i) the first fusion protein comprises a ferritin light chain and an Fab fragment capable of specifically binding to a first antigen, and
(ii) the second fusion protein comprises a ferritin light chain and an Fab fragment capable of specifically binding to a second antigen, or
(b) (i) the first fusion protein comprises N-ferritin and an Fab fragment capable of specifically binding to a first antigen, and
(ii) the second fusion protein comprises C-ferritin and an Fab fragment capable of specifically binding to a second antigen,
wherein, within each fusion protein, the Fab fragment is fused to the N-terminus of the ferritin light chain, the N-ferritin, or the C-ferritin, and
wherein the first antigen is distinct from the second antigen.

98. A nanocage comprising a plurality of first fusion proteins, a plurality of second fusion proteins, and a plurality of third fusion proteins, wherein
(a) the first fusion protein comprises a ferritin light chain and an Fab fragment capable of specifically binding a first antigen,
(b) the second fusion protein comprises a C-ferritin and an Fab fragment capable of specifically binding a second antigen, and
(c) the third fusion protein comprises an N-ferritin and an Fc fragment,
wherein, within each fusion protein the Fab or Fc fragment is fused to the N-terminus of the ferritin light chain, the C-ferritin, or the N-ferritin, and
wherein the first antigen is distinct from the second antigen.

99-114. (canceled)

115. The nanocage of claim 98, further comprising a plurality of fourth fusion proteins, each fourth fusion protein comprising a C-ferritin and an Fab fragment capable of specifically binding a third antigen which is distinct from the first and second antigen, wherein the first, second, and third antigens are associated with HIV-1; and
wherein
the Fab fragment of the first fusion protein is a PDGM1400 Fab,
the Fab fragment of the second fusion protein is a 10E8v4 Fab,
the Fc fragment of the third fusion protein is a human IgG1 Fc fragment, and
the Fab fragment of fourth fusion protein is a N49P7 Fab.

116. The nanocage of claim 98, further comprising a plurality of fourth fusion proteins, each fourth fusion protein comprising a C-ferritin and an Fab fragment capable of specifically binding a third antigen which is distinct from the first and second antigen, wherein the first and second antigens are associated with HIV-1; wherein the third antigen is associated with CD4; and wherein
the Fab fragment of the first fusion protein is a PDGM1400 Fab,
the Fab fragment of the second fusion protein is a 10E8v4 Fab,
the Fc fragment of the third fusion protein is a human IgG1 Fc fragment, and
the Fab fragment of fourth fusion protein is an iMab Fab.

117. A therapeutic or prophylactic composition comprising the nanocage of any one of claim 79.

118. A method of for treating or preventing a disease or condition, the method comprising administering the nanocage the composition of claim 117 to a subject in need thereof.

119. A method of making a multispecific self-assembling nanocage, the nanocage characterized by a pre-selected ratio of different specificities, the method comprising the step of:
co-transfecting a host cell with one or more expression plasmids comprising a plurality of polynucleotides, each polynucleotide encoding a fusion protein,
wherein each fusion protein comprises (i) a nanocage monomer or a subunit thereof and (ii) an antibody or antibody fragment of a given specificity,
wherein the step of co-transfecting comprises co-transfecting the polynucleotides in a ratio based on the pre-selected ratio;
obtaining polypeptides produced by the host cell; and
purifying the polypeptides by affinity selection for all the different specificities to be present in an assembled nanocage.

120. (canceled)
